(19) United States
(12) Patent Application Publication
(10) Pub. No.: US 2013/0281504 A1
(43) Pub. Date: Oct. 24, 2013

(54) Reduction of Microglia-Mediated Neurotoxicity by KCa3.1 Inhibition

(71) Applicants: Heike Wulff, Davis, CA (US); Lee-Way Jin, Davis, CA (US); Izumi Maezawa, Davis, CA (US)

(72) Inventors: Heike Wulff, Davis, CA (US); Lee-Way Jin, Davis, CA (US); Izumi Maezawa, Davis, CA (US)

(73) Assignee: THE REGENTS OF THE UNIVERSITY OF CALIFORNIA, Oakland, CA (US)

(21) Appl. No.: 13/807,245

(22) PCT Filed: Jun. 28, 2011
(86) PCT No.: PCT/US11/42243

§ 371 Date: Jul. 8, 2013

Related U.S. Application Data
(60) Provisional application No. 61/359,318, filed on Jun. 28, 2010.

(51) Int. Cl. A61K 31/415

(52) U.S. Cl. CPC A61K 31/415
USPC 514/406; 548/373.1

(57) ABSTRACT

Methods for deterring microglia-mediated neurotoxicity in a human or non-human animal subjects comprising the step of inhibiting or blocking the intermediate-conductance calcium-activated potassium channel KCa3.1 in microglia, such as in subjects how suffer from neurodegenerative diseases (e.g., Alzeheimer's Disease) or ischemic/anoxic/hypoxic conditions. The inhibition or blocking of the KCa1.3 channels may be accomplished by administering a KCa3.1 inhibiting substance, such as 1-[(2-chlorophenyl)diphenylmethyl]-1H-pyrazole (TRAM-34).

### RELATED APPLICATIONS

This patent application claims priority to U.S. Provisional Patent Application No. 61/359,318, filed Jun. 28, 2010 and is a continuation in part of copending U.S. patent application Ser. No. 11/805,763 filed May 25, 2007, which is a division of U.S. patent application Ser. No. 10/402,532 filed Mar. 28, 2003 and issued as U.S. Pat. No. 7,235,577 on Jun. 26, 2007, which is a division of U.S. patent application Ser. No. 09/479,375, filed Jan. 6, 2000 and issued as U.S. Pat. No. 6,803,375 on Oct. 12, 2004, the entire disclosure of each such application and patent being expressly incorporated herein by reference.

### STATEMENT REGARDING GOVERNMENT SUPPORT

This invention was made with United States Government support under Grant Nos. AG025500 and AG031362 awarded by the National Institutes of Health. The United States Government may have certain rights in the invention.

### FIELD OF THE INVENTION

The present invention relates generally to the fields of chemistry, pharmacology and medicine and more particularly to the treatment of neurodegenerative diseases, deterring or reducing neuronal damage following ischemic/hypoxic/anoxic events and treatment of other conditions wherein microglia-mediated neurotoxicity occurs.

### BACKGROUND OF THE INVENTION

Pursuant to 37 CFR 1.71(e), this patent document contains material, which is subject to copyright protection. The copyright owner does not object to facsimile reproduction of the entire patent document, as it appears in the Patent and Trademark Office patent file or records, but otherwise reserves all copyright rights whatsoever.

Microglia are non-neural, interstitial cells of mesodermal origin that form part of the supporting structure of the central nervous system in humans and other mammals. Microglia are tissue resident macrophages of the brain. Microglia come in various forms and may have slender branched processes. They are migratory and, when activated (usually by some instigating stimulus), can act as phagocytes, which engulf and remover nervous tissue waste products.

In various neurodegenerative diseases, damage to nerve cells is believed to occur, at least in part, due to activation of microglia by some instigating stimulus (an “activator”). For example, in Alzheimer's disease (AD), amyloid plaques accumulate between nerve cells (neurons) in the brain. Amyloid is a term, which broadly refers to protein fragments that the body produces normally. Beta amyloid (Aβ) is a protein fragment that comes from an amyloid precursor protein. In healthy brains, these Aβ protein fragments are broken down and eliminated. However, in AD, the Aβ protein fragments aggregate to form hard, insoluble plaques. Aggregated forms of Aβ as well as soluble precursor forms called oligomeric Aβ act as microglial activators. The activated microglia have a beneficial effect of phagocytiozing Aβ deposits, but they also have deleterious neuron-damaging effects, such as direct microglial neuron killing and by causing production of neurotoxic nitric oxide (NO) and inflammatory cytokines.

Microglia also play a roll in causing brain damage following hypoxic or anoxic insults to the brain. Hypoxic or anoxic brain insults may occur due to various causes, including but not limited to ischemic or hemorrhagic strokes, cardiac arrest and resuscitation, carbon monoxide poisoning, trauma, asphyxiation, strangulation, drowning, hemorrhagic shock, inhalant substance abuse (“huffing”), brain edema, iatrogenic disruption of cerebral circulation during surgery or other medical procedures, etc.

Inhibition of the intermediate-conductance calcium-activated potassium channel KCa3.1 in microglial cells is viewed as a potential therapeutic approach for reducing microglia-mediated neurotoxicity. However, it is desirable for therapies aimed at microglia-mediated neurotoxicity to meet the following goals:
(a) reduce the neurotoxic effects of microglia while at the same time maintaining their neuroprotective functions such as phagocytosis of amyloid-beta deposits;
(b) be specific to microglia so that its inhibition does not adversely affect important neuronal or astroglia functions; and
(c) not be broadly immunosuppressive.
   In this patent application, Applicants describe compositions and methods for reducing microglia-mediated neurotoxicity in a manner that meets some or all of these goals.

### SUMMARY OF THE INVENTIONS

In accordance with the present invention, there is provided a method for deterring microglia-mediated neurotoxicity in a human or non-human animal subject, said method comprising the step of inhibiting or blocking the intermediate-conductance calcium-activated potassium channel KCa3.1 in microglia. The inhibition or blocking of the KCa1.3 channels may be accomplished by administering to the subject a therapeutically effective amount of a KCa3.1 inhibiting substance, non-limiting examples of which are described in U.S. Pat. No. 7,235,577. One such substance comprises 1-[(2-chlorophenyl)diphenylmethyl]-1H-pyrazole (TRAM-34).

Further in accordance with the invention, in some embodiments, the inhibition or blockade of the intermediate-conductance calcium-activated potassium channel KCa3.1 may be carried out in a manner that reduces neurotoxic effects of the microglia without preventing beneficial effects of the microglia.

Still further in accordance with the invention, the method may be carried out to deter or slow neuron damage in subjects who suffer from a neurodegenerative disease. Some such subjects may have Aβ deposits (such as those suffering from Alzeheimer's Disease or who are in the process of developing Alzeheimer's Disease) and the inhibition or blockade of the intermediate conductance calcium-activated potassium channel KCa3.1 may be carried out in a manner that reduces at least one neurotoxic effect of microglia (e.g., microglia-mediated neuronal killing, microglial production of NO and/or microglial cytokine production) while not preventing microglia from phagocytosing Aβ deposits.

Still further in accordance with the invention, in some embodiments, the method will be carried out to reduce neural damage in subjects who have suffered or are suffering an ischemic, anoxic or hypoxic condition, event or insult, such as those who suffer a) ischemic stroke, b) hemorrhagic stroke, c) cardiac arrest and resuscitation, d) carbon monoxide poisoning, e) trauma, f) asphyxiation, g) strangulation, h) drowning, i) hemorrhagic shock, j) inhalant substance abuse or huffing, k) brain edema and l) iatrogenic disruption of cerebral circulation during a surgery or other medical procedure.

Still further aspects and details of the present invention will be understood upon reading of the detailed description and examples set forth herebelow.

### BRIEF DESCRIPTION OF THE DRAWINGS

FIG. 1A is a panel of photomicrographs referenced in Example 1 below.

FIGS. 1B through 1E are graphs referenced in Example 1 below.

FIG. 2A is a panel of photomicrographs referenced in Example 1 below.

FIG. 2B is a Western Blot referenced in Example 1 below.

FIGS. 2C through 2E are graphs referenced in Example 1 below.

FIG. 3 is a graph referenced in Example 1 below.

FIGS. 4A and 4B are graphs referenced in Example 1 below.

FIG. 5A is a graph referenced in Example 1 below.

FIGS. 6A through 6C are panels of photomicrographs referenced in Example 1 below.

FIG. 6D is a graph referenced in Example 1 below.

FIG. 6E is a Western Blot referenced in Example 1 below.

FIG. 7A is a panel of photomicrographs referenced in Example 1 below.

FIG. 7B is a graph referenced in Example 1 below.

FIG. 7C is a panel of photomicrographs referenced in Example 1 below.

FIG. 7D is a Western Blot referenced in Example 1 below.

FIGS. 8A through 8C are graphs referenced in Example 1 below.

FIG. 8D is a panel of photomicrographs referenced in Example 1 below.

FIG. 9 is a graph referenced in Example 1 below.

FIG. 10 is a panel of photomicrographs referenced in Example 2 below.

FIGS. 11A through 11D are graphs referenced in Example 2 below.

FIGS. 12A through 12D are graphs referenced in Example 2 below. The legend above FIG. 12A shows areas in serial brain sections from which samples were obtained.

FIGS. 13A and 13 B are graphs referenced in Example 2 below.

FIGS. 14A through 14C are graphs with associated photomicrographs referenced in Example 2 below.

### DETAILED DESCRIPTION

The following detailed description and the accompanying figures to which it refers are intended to describe some, but not necessarily all, examples or embodiments of the invention. The described embodiments are to be considered in all respects only as illustrative and not restrictive. The contents of this detailed description and the accompanying drawings do not limit the scope of the invention in any way.

### Example 1

### Selective Reduction of Microglia-Medicated Neuroinflammation in AD by Inhibition KCa3.1

To examine microglia activators that are pathologically relevant to the early stage of AD development, Applicants investigated the ability of low levels of Aβ oligomers (AβO) to activate microglia and discovered, in the course of such work, that AβO can activate microglia at concentrations in the range of 5-50 nM. Such low concentrations of AβO are usually not sufficient to cause direct neurotoxicity. This effect follows a bell-shaped dose-response curve and tapers off at concentrations above 100 nM. Furthermore, Applicants have determined that AβO-activated microglia release soluble neurotoxic substances that cause neuronal damage, and this mode of microglia activation and neurotoxicity is dependent on microglial KCa3.1. This indicates that neuroinflammation mediated by microglia is a significant contributor to early AD pathogenesis when Aβ starts to accumulate in brain prior to fAβ deposition.

### A. Experimental Procedures

Chemicals—

Lipopolysaccharides (LPS), Congo red (CR), 3-(4,5-dimethylthiazol-2-yl)-2,5-diphenyltetrazolium bromide (MTT), polyinosinic acid (poly I), [3,8-diamino-5-(3-(diethylmethylamino)propyl)-6-phenyl phenanthridinium diiodide (PI), apamin, and doxycycline were purchased from Sigma (St. Louis, Mo.). The CD40 ligand, CD 154, was purchased from Santa Cruz Biotechnology Inc. (Santa Cruz, Calif.). The macrophage colony stimulatory factor (MCSF) was purchased from R&D Systems. The KCa3.1 inhibitor TRAM-34 (1-[(2-chlorophenyl)diphenylmethyl]-1H-pyrazole) was synthesized as described in expressly incorporated U.S. Pat. Nos. 6,803,375 and 7,235,577.

Preparation of Aβ Solutions—

AβO solutions as well as the unaggregated and fibrillary Aβ were prepared as described. Our preparation of AβO follows the procedure described by Lambert et al.; (1998) Proc Natl Acad Sci USA 95, 6448-6453, except that the Aβ42 peptide was diluted with Opti-MEM culture medium instead of the F12 medium originally described, before incubation at 4° C. for 24 h to generate oligomers. This preparation of AβO has been extensively characterized in our laboratory. To ensure consistency of quality, a random sample from each batch was chosen and imaged using electron microscopy and atomic force microscopy to characterize the size and shape of the aggregates. The biological activities of each batch were confirmed by determining AβO's neurotoxic activity, synaptic binding activity and ability to rapidly induce exocytosis of MTT formazan.

Soluble AβO from Human Brain Tissue—

Hippocampal tissue samples were obtained postmortem from three AD subjects and two cognitively and pathologically normal control subjects. A11 subjects had comparable postmortem intervals averaging 5.5 h. Soluble extracts from brain tissues were prepared as described by Lacor, P. N., et al.: (2004) J Neurosci 24, 10191-10200. Molecular weight fractionation of oligomeric species was performed using Centricon YM-100 and YM-10 concentrators (Millipore, Bedford, Mass., USA). The relative abundance of AβO in the resulting solutions was determined by Western blots using the 6E10 antibody and dot blots using the A11 antibody. While the AD samples contained various amounts of AβO, the two control samples showed no detectable AβO on Western blots and almost background levels on dot blots.

To ensure accurate measurement of the total quantity of Aβ in the extract, Applicants used two Aβ ELISA kits purchased from IBL America (Minneapolis, Minn.) and Wako Chemicals USA (Richmond, Va.), respectively. The procedures were conducted according to manufacturer's protocols.

Primary Cultures—

Primary microglia cultures derived from newborn C57BL/6J mice were prepared from mixed glia cultures with the “shaking off” method described by Maezawa, I., et al.; (2006) FASEB J 20, 797-799. Cultures were ≧99% pure for microglia as demonstrated by anti-IBA1 immunostaining. To obtain conditioned medium (CM) for treating neurons, microglia were first cultured in 24-well culture plates at a density of 1.3×10⁵ cells/cm² for 24 hrs in DMEM with 10% fetal bovine serum (DMEM10). Cultures were washed extensively and changed to the Neurobasal medium with B27 supplement (NB/B27, Invitrogen) without serum and cultured for another 24 hrs. The NB/B27-based microglia CMs were collected, briefly centrifuged, and used immediately or frozen for future uses.

Hippocampal neuronal cultures were prepared from newborn wild type C57BL/6J mice according to the method of Xiang et al (26). Neurons were cultured in NB/B27 at a density of 2.5×10⁵ cells/well in 12-well plates or 8×10⁵ cells/well in 6-well plates for at least 14 days before they were treated with microglia CM.

Hippocampal slice cultures (400 μm-thick) were prepared from 7-day-old C57BL/6J mice as previously described (25) and cultured for 10 days in vitro before use. Neuronal damage in the slices was monitored by PI uptake following Bernardino et al (27). PI itself is not toxic to neurons (27). Briefly, hippocampal slices were pretreated with or without doxycycline (20 μM) or TRAM-34 (1 μM) for 1 hr and then treated with medium containing PI (2 μM) and AβO of indicated concentration, with or without doxycycline or TRAM-34. After 24 hrs, the slices were observed under a Nikon Eclipse E600 microscope and the red (630 nm) fluorescence emitted by PI taken up by damaged cells was photographed by a digital camera (SPOT RTke, SPOT Diagnostics, Sterling Heights, Mich.) with fixed exposure time.

BrdU Incorporation Assay—

Microglia were plated onto 48-well culture plates at a density of 1×10⁵ cells/well in DMEM10 and incubated for 24 hrs. Cells were washed with serum-free Opti-MEM medium three times and treated with indicated concentrations of AβO in Opti-MEM. After 5 hr incubation, 10 μM BrdU was added and allowed to be incorporated into DNA of proliferating cells during an additional 16 hr of incubation. Cells with positive BrdU incorporation were determined by an immunocytochemical stain for BrdU using an anti-BrdU antibody conjugated with Alexa594 (Invitrogen) following the manufacturer's protocol (Chemicon), and counted.

NFκB Assay—

The detection of cells with active NFκB was performed according to the method described by Franciosi, S., et al.; (2006) J Neurosci 26, 11652-11664. Briefly, microglia were plated as described above and treated with activators for 2 hrs. Cells were fixed and stained with anti-NF-κBp65 antibody (1:250, Chemicon) and with the nuclear dye DAPI.

Assays for Neuronal Viability—

Hippocampal neurons were prepared as described above and were plated onto 96-well plate at a density of 6×10⁴ cells/well and cultured for 14 days. CM from microglia cultures was added onto neurons with indicated dilutions, and cultures were incubated for 24 hrs. Neuronal viability was evaluated by the MTT assay and the LDH release assay as previously described by Maezawa, I., et al.; (2006) J Neurochem 98, 57-67 and Maezawa, I., et al.; (2006) FASEB J 20, 797-799.

Immunofluorescence Staining and Quantification—

For immunofluorescent staining of neurons, cultures were fixed in 4% paraformaldehyde and stained with anti-PSD95 (1:200, Cell Signaling), anti-MAP2 (1:500, Chemicon) and anti-acetylated α-tubulin (1:250, Zymed). For immunofluorescent staining of microglia, cultures or hippocampal slices were fixed in 4% paraformaldehyde and stained with anti-IBA1 (1:500, Wako Chemicals USA), anti-SRA (E-20, 1:200, Santa Cruz Biotechnology), and anti-CD11b (1:200, AbD Serotec USA, Raleigh, N.C.). The fixed cells or hippocampal slices were incubated with antibodies for overnight at 4° C. followed by secondary Alexa488 conjugated anti-mouse or Alexa568 conjugated anti-rabbit antibody (1:700, Molecular probes). Immunostained images were observed under a Nikon Eclipse E600 microscope and photographed by a digital camera (SPOT RTke, SPOT Diagnostics, Sterling Heights, Mich.).

For quantification of the PSD95-immunoreactive puncta along the dendrites, photomicrographs of PSD95 immunostained cultures were randomly taken from each culture condition. The images were transformed to 8 bit gray scale and analyzed with the Image J program. The number of puncta in each photomicrograph was counted and normalized by dendritic length. The photography and analysis were conducted in an investigator-blinded manner

Western Blot Analysis—

To obtain lysates, cells or tissues were washed with ice-cold PBS and incubated with a buffer containing 50 mmol/L Tris-HCl (pH 7.4), 150 mmol/L NaCl, 2% SDS, proteinase inhibitor cocktail (Sigma), and phosphatase inhibitor cocktail (Sigma). Lysates were briefly sonncated and cleared by centrifugation at 50,000 rpm for 10 min. Equivalent amounts of protein were analyzed by Tris/HCL gel electrophoresis. Proteins were transferred to polyvinylidene difluoride membranes and probed with antibodies. Visualization was performed using enhanced chemiluminescence (ECL, Amersham Pharmacia, Piscataway, N.J.).

The following primary antibodies (dilutions) were used: anti-p38MAPK (1:1000, Cell Signaling Technology, Boston, Mass.), anti-phospho-p38MAPK (1:1000, Cell Signaling Technology), anti-Synaptophysin (1:1000, Abcam), anti-PSD95 (1:1000, cell signaling), anti-GRIP 1 (1:1000, UpState), anti-MAP2 (1:1000, Chemicon), anti-acetylated α-tubulin (1:2000, Zymed), and anti-β-actin (1:3000, Sigma). Secondary antibodies were HRP-conjugated anti-rabbit, anti-goat, or anti-mouse antibody (1:3000 Amersham).

Assay for Nitric Oxide (NO)—

Conditioned medium collected from microglia cultures (1×10⁵/0.75 cm²) and hippocampal slices treated with Aβ for 24 hrs were analyzed by the Nitric Oxide Quantization Kit according to the protocol of the manufacturer (Active Motif, Carlsbad, Calif.). Data were normalized to the amount of total protein.

Patch-Clamp Experiments—

Microglia, that were “floating off” from their feeding astrocyte layer, were harvested with a pipette, washed, attached to poly-L-lysine coated cover-slips, and then studied in the whole-cell mode of the patch-clamp technique with an EPC-10 HEKA amplifier. The pipette solution contained 145 mM K⁺ aspartate, 2 mM MgCl₂, 10 mM HEPES, 10 mM K₂EGTA and 8.5 mM CaCl₂ (1 μM free Ca²⁺), pH 7.2, 290 mOsm. To reduce chloride “leak” currents, we used a Na⁺ aspartate external solution containing 160 mM Na⁺ aspartate, 4.5 mM KCl, 2 CaCl₂, 1 mM MgCl₂, 5 mM HEPES, pH 7.4, 300 mOsm. K⁺ currents were elicited with voltage ramps from −120 to 40 mV of 200 ms duration applied every 10 s. Whole-cell KCa3.1 conductances were calculated from the slope of the TRAM-34 sensitive K_{Ca} current between −80 mV and −75 mV where KCa3.1 currents are not “contaminated” by Kv1.3 (which activates at voltages above −40 mV) or inward-rectifier K⁺ currents (which activate a voltages more negative than −80 mV). Cell capacitance, a direct measurement of cell surface area, was continuously monitored during recordings. KCa3.1 current density was determined by dividing the TRAM-34-sensitive slope conductance by the cell capacitance.

Statistical Analysis—

Statistical analyses were performed using SigmaPlot 11 software (Systat Software, Inc). Analysis of variance (ANOVA) was used to compare quantitative values from cultures across groups. Tukey's studentized range test was used to adjust for multiple comparisons in post-hoc pairwise tests.

### B. Results

AβO at Low Nanomolar Concentrations Stimulates Microglia into a Distinct Activation Phenotype.

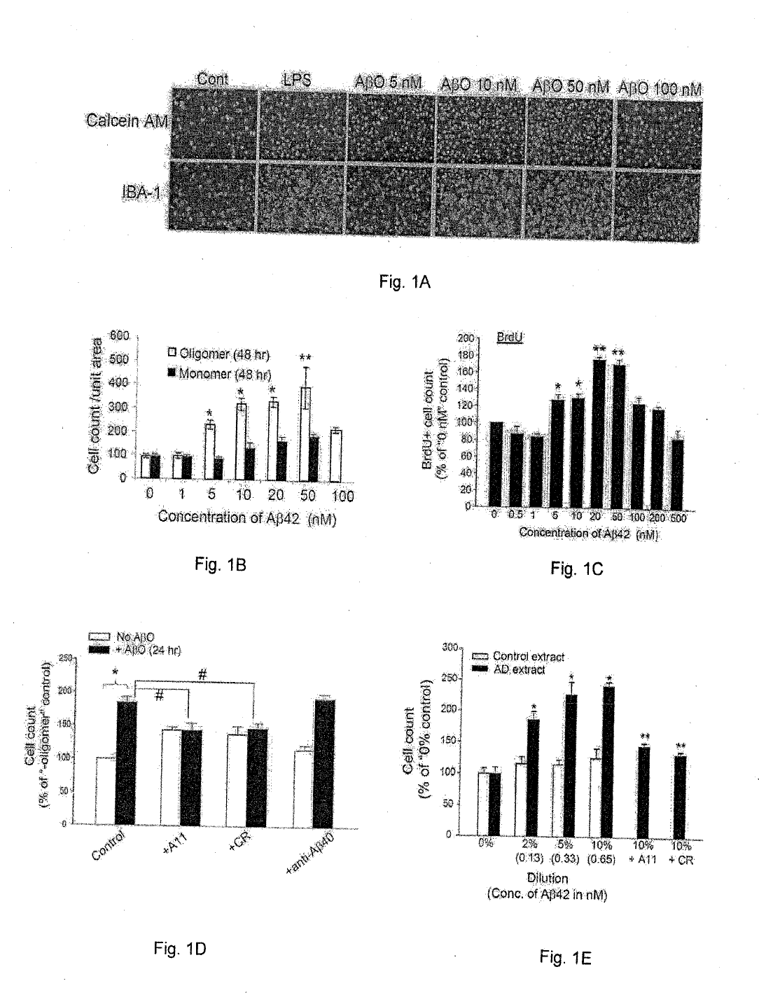

Our AβO preparations affected viability of cultured hippocampal neurons at concentrations above 100 nM. The same preparations induced proliferation of cultured microglia, starting at 5 nM (22.5 ng/ml) and maximizing at around 50 nM (225 ng/ml), as seen in FIGS. 1A and 1B, thereby demonstrating that synthetic and human-derived AβO activate microglia proliferation at sub-neurotoxic concentrations. Specifically, in FIG. 1A, Microglia were treated with AβO of indicated Aβ concentrations for 48 hrs and the cell numbers determined. AβO treatment caused microglia proliferation in a dose-dependent manner and the effect tapered off at 100 nM. Aβ monomer caused a mild but statistically not significant increase of proliferation. Data presented are means±S.E. n=3, * p<0.05 and ** p<0.001 compared with the “0 nM”, solvent treatment controls. In FIG. 1B, all cells in the microglia cultures were stained with Calcein AM. The identity of cells was confirmed by immunostaining with IBA-1, a microglia marker.

At 24 hr and 48 hr post-stimulation, cell counts in cultures treated with 20 nM AβO were 184±8.59% (mean±S.E, n=6, p<0.001, see FIG. 1D) and 330±21.8% (n=3, p<0.001, see FIG. 1B) of the values of solvent-treated controls, respectively. The identity of the proliferating cells was confirmed by their immunoreactivity to anti-ionized calcium binding adaptor molecule 1 (IBA-1), a microglia specific marker (FIG. 1A). The mitogenic effect was confirmed by 5′-bromodeoxyuridine (BrdU) incorporation, as shown in FIG. 1C. AβO treatment caused a dose-dependent incorporation of BrdU, following a bell-shaped curve. n=3,* p<0.05 and ** p<0.001 compared with the “0 nM”, solvent treatment controls. Proliferation measured by both cell counting and BrdU followed a bell-shaped curve (FIGS. 1B and 1C), which is similar to previously reported chemotactic activity of soluble Aβ for macrophages/microglia that also maximized at low nanomolar concentrations.

As shown in FIG. 1D, the mitogenic effect was blocked by a neutralizing oligomer-specific antibody A11 and by Congo red (CR), an amyloid ligand known to neutralize the activity of AβO. Specifically, with respect to FIG. 1D, Microglia were treated for 24 hrs with 20 nM AβO or mock-treated with solvent, in the presence of A11 (50 nM) or CR (100 nM), and cell numbers were determined. n=5, * p<0.001 and # p<0.05. Since both A11 and CR recognize conformational features, but not the primary sequence of Aβ, these results indicate that the conformation of AβO was responsible for the mitogenic effect. The unaggregated Aβ1-42 showed a mild although statistically not significant induction of proliferation (FIG. 1B), attributable to the small amount of AβO invariably formed in aqueous solution since this effect was also blocked by A11 and CR. Fibrillar Aβ up to 1 μM and preparations of reverse-sequence Aβ peptide showed no effect. These results indicate that at concentrations lower than those required for direct neurotoxicity, AβO is mitogenic for microglia.

To determine if AβO derived from human brain would also promote microglia proliferation, we obtained soluble extracts from hippocampi of AD and age-matched non-demented control subjects and prepared the fractions containing AβO but excluding unaggregated Aβ or large aggregates. This preparation of human AβO has been well characterized in previous studies. The quantities of Aβ in these extracts were determined by sandwich ELISA. Because ELISA may underestimate the total quantity of Aβ when in oligomeric forms, we first deaggregated Aβ in the extract with guanidine HCl as previously described. In addition, Applicants used two different commercial ELISA kits and obtained consistent Aβ concentrations. Surprisingly, soluble extracts containing AβO from all three AD brains consistently stimulated microglia proliferation at sub- to low nanomolar concentrations (˜0.11 to 1.24 nM Aβ42) (FIG. 1E) and thus were about 50-fold more potent than the synthetic AβO. Specifically, with respect to FIG. 1E, E. Soluble extracts with a 10-100 kDa MW cutoff were obtained from 3 AD and 2 control hippocampi. Microglia were treated with these extracts diluted into the Opti-MEM medium at indicated percentile (v/v) and the numbers of cells were determined after 24 hrs. Shown are data from using a representative pair of AD (black bars) and control (white bars) extracts. The actual Aβ42 concentrations (in nM) for the indicated dilutions of the AD extract are indicated in the x-axis. n=3, * p<0.05 for control vs AD in each concentration between the pair of extracts, and **p<0.001 for “10% AD” vs “10% AD+A11” and for “10% AD” vs “10% AD+CR”. Parallel treatments of hippocampal neurons with soluble AD brain extracts of the same concentrations did not show any effect on neuronal viability. The equivalent fractions from two age-matched control individuals contained little Aβ and showed no microglia mitogenic effect. The addition of A11 or CR blocked the mitogenic effect (FIG. 1E), confirming that this effect was mediated by AβO. These results again show that AβO is a potent mitogen for microglia at concentrations that do not cause direct neurotoxicity.

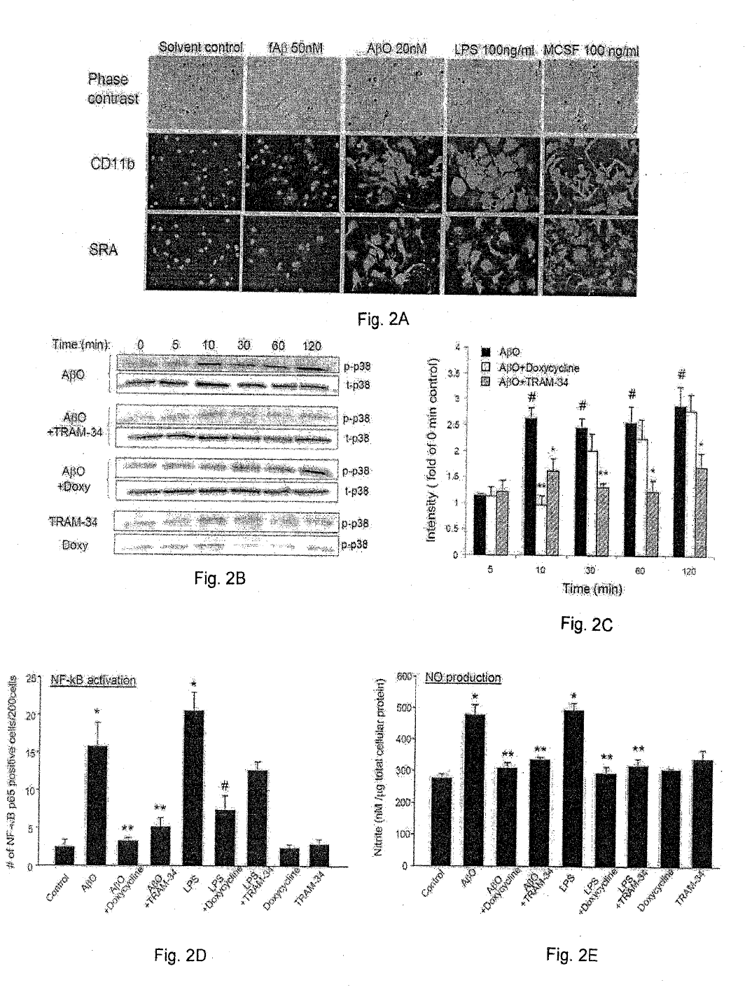

As shown in FIG. 2A, microglia after synthetic or AD patient brain-derived Aβ treatment showed a morphology resembling those activated by lipopolysaccharides (LPS) or macrophage colony stimulatory factor (MCSF). Specifically, Phase contrast photomicrographs of microglia treated for 24 hrs with solvent, 20 nM AβO, 100 ng/ml LPS, and 100 ng/ml MCSF, respectively. Biochemical and immunocytochemical evidence also supports that AβO treatment induces an activated phenotype. AβO-treated microglia showed strong up-regulation of MAC-1 (CD11b) and scavenger receptor A (SRA), two markers of microglia activation (FIGS. 2B and 2C), and increased levels of phosphorylated, therefore activated, p38 mitogen activated protein kinase (p38MAPK) (225±38% of vehicle treatment control, n=3, p<0.001), while the total levels of p38MAPK protein were not changed (FIG. 2D). AβO treatment further induced a six-fold increase of cells showing immunoreactivities of active nuclear factor κB (NFκB, n=6, p<0.001, FIG. 2E). These intracellular responses are qualitatively similar to those induced by LPS activation through the CD14/TLR4 co-receptors, including the stimulation of pathways that are dependent on p38MAPK and NFκB signaling. More specifically, with respect to FIG. 2B, Immunofluorescent staining for CD11b of microglia with indicated treatment for 24 hrs. With respect to FIG. 2C, immunofluorescent staining for SRA of microglia with indicated treatment for 24 hrs. In FIG. 2D, microglia were treated for 30 minutes and the activation state of p38MAPK was evaluated by Western blot using an antibody for its phosphorylated epitope. An antibody for p38MAPK was used to quantify the total p38MAPK level. The activation of p38MAPK is represented by the band intensity of phosph-p38MAPK normalized to that of total p38MAPK. Quantification from three independent experiments showed a 2.25 fold increase in p38MAPK phosphorylation after Aβ treatment (p<0.001). There was no significant difference between “control” vs “AβO+TRAM-34” and “control” vs “LPS+TRAM-34”. The concentrations used are: LPS, 100 ng/ml; doxycycline, 20 μM; TRAM-34, 1 μM. In FIG. 2E, after indicated treatment for 2 hrs, the microglia were immunostained with an antibody for p65 of NFκB to mark cells with NFκB activation. Numbers of p65-immunoreactive cells per 200 DAPI labeled cells were determined. Treatment with Aβ and LPS increased the number of cells with activated NFκB (n=6, * p<0.001 compared with control). The AβO-induced activation was prevented by 20 μM doxycycline and 1 μM TRAM-34 (** p<0.001 compared with the “AβO” group). LPS-induced activation was prevented by doxycycline (# p<0.001 compared with the “LPS” group), but not significantly by TRAM-34. In FIG. 2F, nitric oxide (i.e., nitrite) production by microglia after 24 hr of indicated treatment was measured in the conditioned medium and normalized by the amount of total cellular protein in each culture. Treatment with Aβ and LPS increased microglia NO production (n=4, * p<0.001 compared with control). The AβO-induced increase was prevented by 20 μM doxycycline and 1 μM TRAM-34 (n=4, ** p<0.001 compared with the “AβO” group). Doxycycline or TRAM-34 alone did not affect NO production.

As mentioned above, microglia activation is often accompanied by increased release of nitric oxide (NO), synthesized by the inducible NO synthase (iNOS). However, Applicants have determined that AβO treatment significantly increased NO generation as evaluated by measuring the concentration of nitrite, its stable metabolite, released into the medium. After normalization to total amount of cellular protein, the data indicate a ˜80% increase of NO release per cell; therefore this increase can not be explained solely by AβO-induced cell proliferation (FIG. 2F). Parallel treatment with iv monomer or fibril failed to show any increase in NO. Besides NO, however, our initial investigation of several commonly studied neuroinflammatory mediators did not show any AβO-induced increases above basal, sometimes undetectable, levels. These mediators include prostaglandin E2, glutamate, and proinflammatory cytokines such as tumor necrosis factor-α (TNF-α), interleukin 1-β (IL1-β), and interleukin 6 (IL6). Parallel microglia cultures treated with LPS or CD40 ligand (CD154) showed variably but significantly increased releases of all of these mediators. These results suggest that Aβ induces a “milder” microglia activation that is significantly different from LPS induced activation.

AβO-Stimulated Microglia Activation Depends on SRA and can be Blocked by Doxycycline.

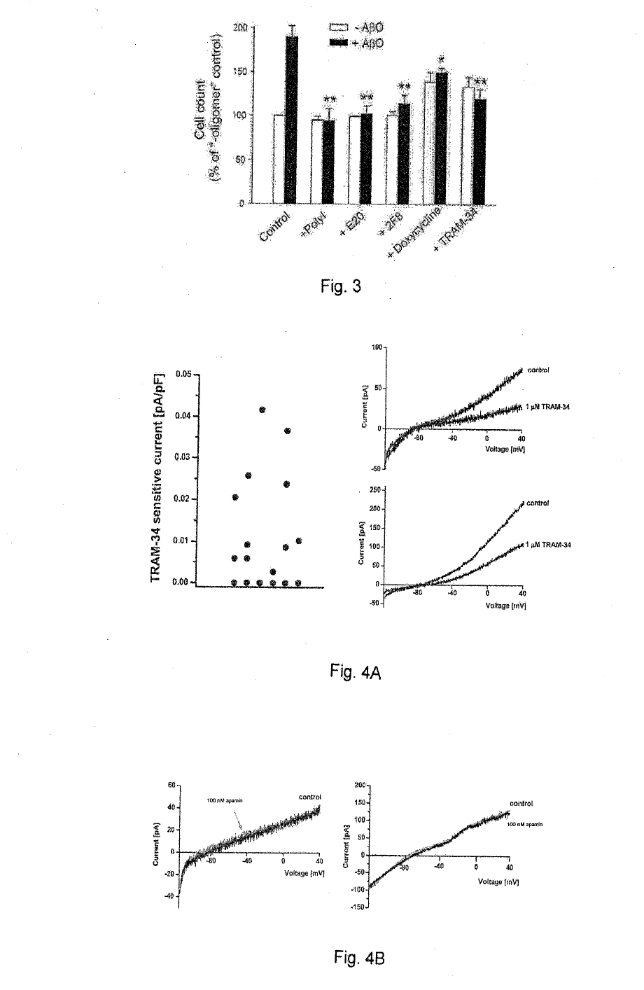

To explore the molecular mechanisms and potential pharmacological blockage of AβO-induced microglia activation, Applicants tested several known inhibitors of microglia activation. It has previously been reported that scavenger receptors, in particular SRA, participate in the binding of fAβ. Because AβO treatment increased microglial expression of SRA (FIG. 2C), it is possible that SRA may recognize the amyloid conformation and mediate at least some of the effects of AβO. Both the general scavenger receptor inhibitor polyinosinic acid (poly I) and two anti-SRA antibodies, E-20 and 2F8 blocked the Aβ-induced microglia activation, while non-specific antibodies of the same immunoglobulin species did not (FIG. 3), suggesting that SRA mediates the interaction of Aβ with microglia. More specifically, in the showing of FIG. 3, Microglia were treated with (black bars) or without (white bars) 20 nM AβO for 24 hrs in the presence of indicated inhibitors: 100 ng/ml poly I, 10 μg/ml anti-SRA antibody E-20, 20 μM doxycycline, or 1 μM TRAM-34. n=4, * p<0.05 and ** p<0.001 compared with the “+AβO control” group. This indicates that AβO-induced microglia activation was blocked by certain inhibitors.

Doxycycline, a second generation tetracycline, has been shown to provide neuroprotection in various models of neuronal injuries by inhibiting microglia activation. Applicants found that doxycycline was able to inhibit AβO-induced microglia activation, including NFκB activation and NO generation (FIG. 3, 2E, 2F). Doxycycline also blocked microglia activation induced by human brain AβO. Interestingly, doxycycline did not suppress p38MAPK phosphorylation following stimulation by either AβO or LPS (FIG. 2D).

Regulation of AβO-Stimulated Microglia Activation by KCa3.1.

By modulating Ca²⁺ signaling, the calcium-activated potassium channel KCa3.1 has been reported to be involved in microglia proliferation, oxidative burst, NO production and microglia mediated neuronal killing in rat microglia. To determine if mouse microglia also express functional KCa3.1 channels, Applicants studied their K⁺ channel expression in the whole-cell configuration of the patch-clamp technique. Similar to previous electrophysiological studies of mouse microglia cultured under various conditions, Applicants observed K_{Ca}, K_{V} and Kᵢᵣ currents. The K_{V} currents exhibited the typical biophysical properties of Kv1.3. K_{Ca} currents became visible when microglia were dialyzed through the patch-pipette with 1 μM of free Ca²⁺ (FIG. 4A). These currents were clearly not carried by the large-conductance K_{Ca} channel KCa1.1 (a.k.a. BK or maxi-K) since they were voltage-independent as determined by ramp-pulses ranging from −100 to +100 mV. The currents further proved insensitive to 100 nM of the KCa2 channel inhibitor apamin (FIG. 4B). However, most of the voltage-independent current component visible during application of voltage-ramps from −120 to +40 mV (FIG. 4A) was sensitive to the selective KCa3.1 blocker TRAM-34. The current remaining after perfusion of 1 μM TRAM-34 probably constitutes a combination of Kv1.3, Kᵢᵣ and another uncharacterized K current. On average mouse microglia exhibited a KCa3.1 current density of 0.026 pA/pF (FIG. 4A). More specifically, In FIG. 4A, the scatter plot of TRAM-34 sensitive (=KCa3.1) current density and two representative recordings shows the effect of 1 μM TRAM-34. In FIG. 4B, the K_{Ca} current is insensitive to the KCa2 channel blocker apamin. These data indicate that mouse microglia do express the calcium-activated K⁺ channel KCa3.1.

Using TRAM-34 as a test compound and pharmacological tool, Applicants probed the role of KCa3.1 in AβO-induced microglia activation and neurotoxicity. TRAM-34 alone did not affect microglia viability, as judged by cell count and NO generation (FIGS. 2F and 3). TRAM-34 did block AβO-induced microglia proliferation, p38MAPK phosphorylation, NFκB activation, and NO generation (FIG. 3, 2E, 2F and supplemental FIG. 3), suggesting that the Aβ effect requires Ca²⁺ signals maintained by KCa3.1-regulated K⁺ efflux. TRAM-34 also blocked microglia activation induced by human brain AβO. Also, TRAM-34 reduced AβO-induced, but not LPS-induced, NFκB activation (FIG. 2E), indicating that different signaling pathways are stimulated by Aβ and LPS.

Aβ Cause Indirect Neuronal Damage Via Microglia.

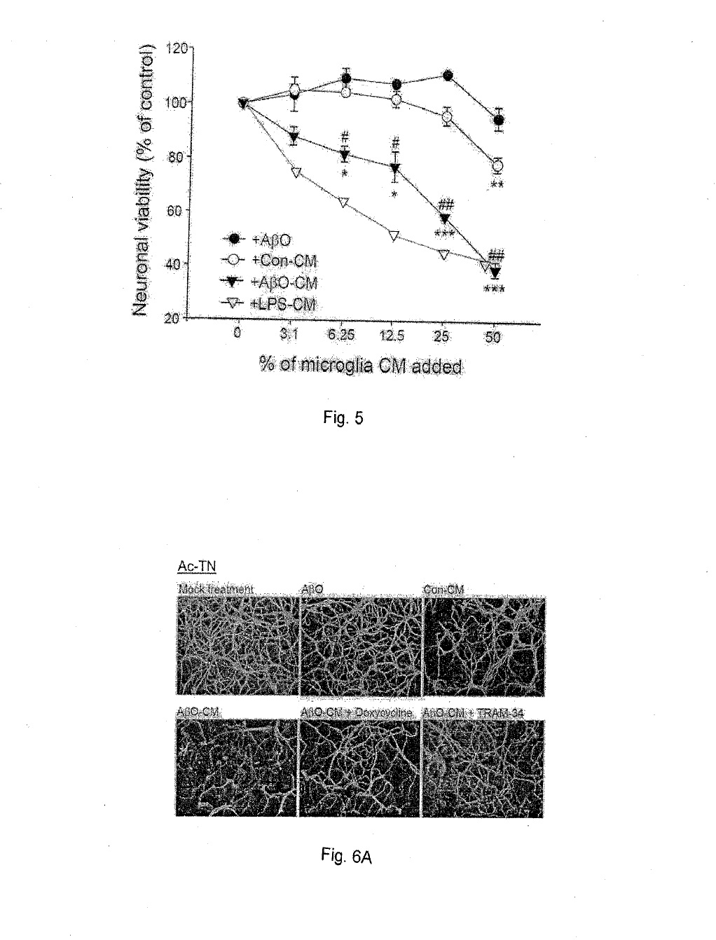

Although AβO induced microglia activation at concentrations lower than those required for direct neurotoxicity, Applicants asked if this activation would result in indirect neuronal damage. To test this possibility, Applicants activated cultured microglia using sub-neurotoxic concentrations of AβO, and transferred the microglia-conditioned medium (AβO-CM) to cultured hippocampal neurons. As controls, Applicants also treated neurons with CM derived from microglia mock-treated with solvent (Con-CM), and medium with Aβ but without being conditioned by microglia. FIG. 5 shows that AβO-CM significantly reduced neuronal viability in a dose-dependent manner. Con-CM also caused mild neurotoxicity at high concentrations, but the toxicity was much less than that exerted by AβO-CM. In contrast, the equivalent amount of AβO directly added to the medium without being conditioned by microglia did not reduce neuronal viability. More specifically, in the showing of FIG. 5, hippocampal neurons, 14-day in vitro, were treated with three kinds of media diluted into NB/B27 culture medium at indicated percentiles. The media were as follows: (1) medium with direct addition of AβO (20 nM) but without conditioned by microglia (+AβO), (2) medium previously conditioned by unstimulated microglia (+Con-CM), and (3) medium previously conditioned by AβO (20 nM)-stimulated microglia (+AβO-CM). Neuronal viability was evaluated by the MTT assay. The data summarized in FIG. 5 show that AβO caused indirect, microglia-mediated neurotoxicity. Independent experiments using the LDH release assay for cell death showed similar results. This indirect neurotoxic effect of AβO is still significant even after normalization by microglia cell number, which increased about 1.8 fold after Aβ treatment. The AβO-CM-mediated neurotoxicity was not blocked by adding A11 or CR to the neuron cultures (data not shown), further supporting that it was not due to residual AβO in CM.

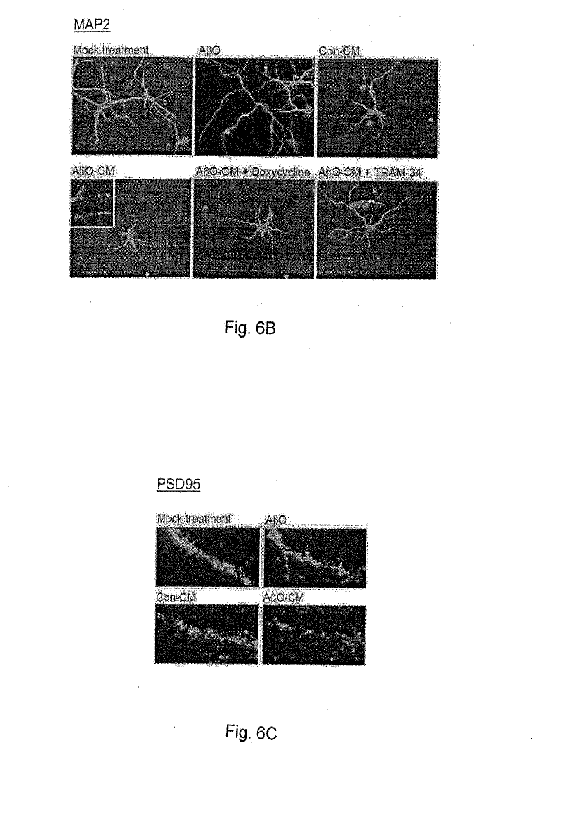

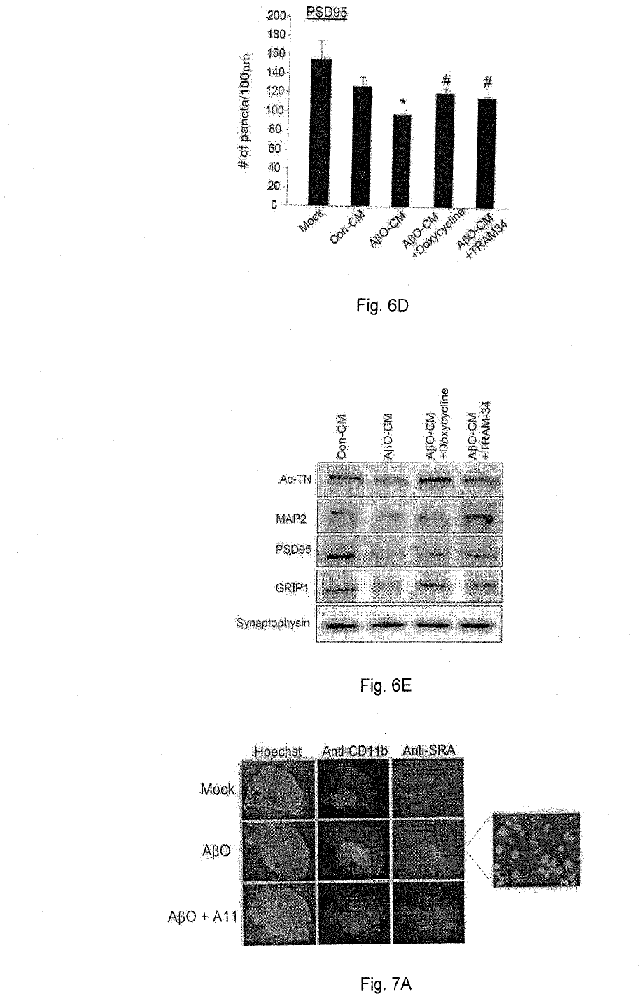

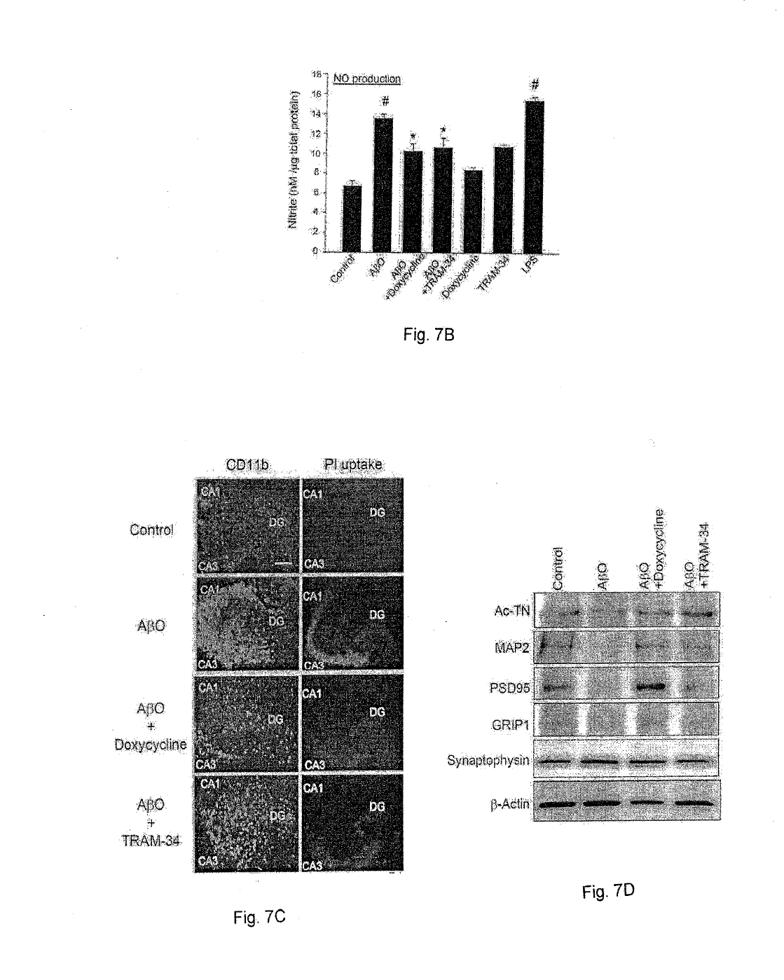

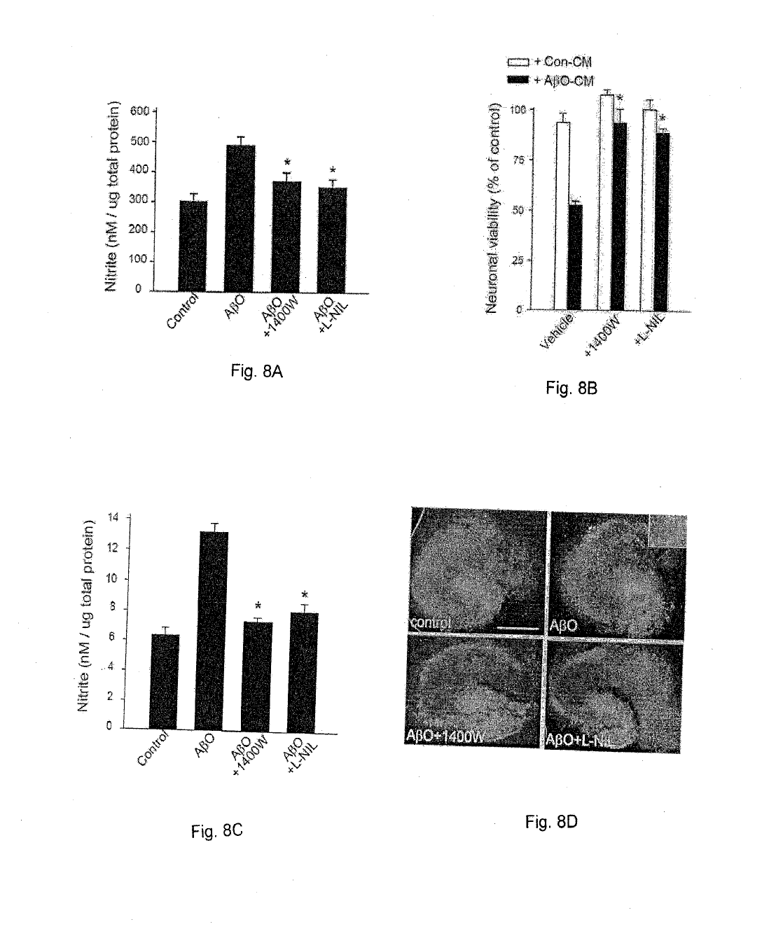

Because dendritic and synaptic damage likely play a more important role than neuronal loss in early stages of AD, Applicants asked if AβO could enhance the ability of microglia to damage dendrites and synapses. Applicants found that neurons treated with AβO-CM showed much more robust signs of dendritic damage than those treated with Con-CM, as demonstrated by immunofluorescent stains with two dendritic markers, acetylated tubulin (Ac-TN) and microtubule associated protein 2 (MAP2) (FIGS. 6A and 6B). Dendrites of AβO-CM-treated neurons were thinner and shorter, with significantly stunted arborization and frequently fragmented or “beaded” appearance, best illustrated by sparsely plated neurons (FIG. 6B). Treatments with AβO-CM also substantially reduced the number of PSD95-immunoreactive puncta, which represent the post-synaptic density (PSD) of excitatory synapses (FIGS. 6C and 6D). Western blots of neuronal extracts revealed that treatments with AβO-CM resulted in reduced levels of Ac-TN, MAP2, PSD95, and another scaffolding protein in the postsynaptic compartment called glutamate receptor interacting protein 1 (GRIP1), but not the presynaptic marker protein synaptophysin (FIG. 6E). In contrast, adding the low nanomolar amount of AβO directly to the hippocampal neuron cultures did not cause a significant reduction of postsynaptic proteins (FIG. 6A-C). More specifically, FIG. 6A dendrites were demonstrated by immunostaining for Ac-TN. In FIG. 6B, dendrites of the sparsely plated neurons were demonstrated by immunostaining for MAP2. The inset of FIG. 6B shows a magnified image wherein “beaded” appearance of dendrites of AβO-CM-treated neurons is visible. FIG. 6C shows PSD95-immunoreactive puncta along representative segments of dendrites. FIG. 6D shows mean counts of PSD95-immunoreactive puncta per unit (100 μm) length. AβO-CM treatment significantly reduced the PSD95 count (n=3, * p<0.001 compared with the “Con-CM” group). This reduction was prevented by inhibition of microglia activation by 20 μM doxycycline or 1 μM TRAM-34 (n=3, # p<0.001 compared with the “AβO-CM” group). FIG. 6E shows a Western blot analysis of lysates from neurons with indicated treatment, analyzed by antibodies to dendritic proteins Ac-TN and MAP2, postsynaptic proteins PSD95 and GRIP1, and presynaptic protein synaptophysin. From the data shown in FIGS. 6A through 6E, it is concluded that Aβ caused indirect, microglia-mediated damage to dendrites and synapses. The levels of dendritic and synaptic markers were compared between hippocampal neurons treated with solvent only (mock treatment), 20 nM AβO, Con-CM, AβO-CM (see FIG. 5), or CM from microglia cultures in which AβO-induced activation was inhibited by doxycycline (AβO-CM+Doxycycline) or TRAM-34 (AβO-CM+TRAM-34), for 24 hrs. A11 CM used were diluted at 25% into the neuronal culture medium.

To confirm that the indirect neurotoxicity was a consequence of microglia activation by AβO, Applicants tested if blocking AβO-induced microglia activation by doxycycline or TRAM-34 also blocks the indirect neurotoxicity. CM from AβO-treated microglia co-incubated with doxycycline or TRAM-34 did not alter the dendritic morphology (FIGS. 5A and 5B) and did not cause significant decreases in levels of Ac-TN, MAP2, PSD95, and GRIP1 (FIGS. 5D and 5E). The lack of KCa3.1 expression in neurons (44, 51-52) makes it unlikely that the above neuroprotective effect was due to a direct effect of residual TRAM-34 in transferred CM on neurons. Taken together, these results indicate that Aβ at sub-neurotoxic concentrations damage dendrites and synapses by an indirect, microglia-mediated mechanism.

AβO Induces Neurotoxicity Via Activating Microglia in Organotypic Hippocampal Slices.

Applicants further tested the AβO effect on microglia using organotypic hippocampal slice cultures, which would better reflect the conditions in the brain in turns of microglial density and their interaction with astroglia and neurons. Treating hippocampal slices with 5-50 nM Aβ caused a significant increase in cells immunoreactive for SRA and CD11b, markers for activated microglia (FIG. 7A). Aβ treatment resulted in a transfaunation of microglia into an activated morphology with retracted processes and enlarged cell bodies (FIG. 7A). Microglia activation was blocked by co-treatment with A11 antibody, confirming that this effect was due to Aβ (FIG. 7A). The CM after the treatment contained increased levels of NO (FIG. 7B). To determine if the neurotoxic effects of low nanomolar Aβ were indeed mediated by activation of microglia, doxycycline and TRAM-34 were tested for their ability to block toxicity to neurons. AβO-induced microglia activation (FIG. 7C), NO generation (FIG. 7B), PI uptake (FIG. 7C), and reduction of dendritic and post-synaptic proteins (FIG. 7D) were substantially ameliorated by co-treatment with either inhibitor. AβO treatment did not increase the release of glutamate, prostaglandin E2, TNF-α, IL1-β, or IL6, consistent with our findings using dissociated microglia cultures. Parallel treatments with unaggregated and fibrillar Aβ did not induce microglia activation. The uptake of the fluorescent dye propidium iodide (PI) is frequently used to monitor neuronal damage in slice cultures. Aβ (20 and 50 nM) treatment resulted in significantly increased uptake of PI by neurons of all hippocampal subfields, a pattern approximating the pattern of microglia activation, although the distribution of activated microglia was broader, extending outside the neuronal domains (FIG. 7C). Western blotting demonstrated that AβO treatment also resulted in significant damages to post-synaptic elements, as demonstrated by reduced levels of Ac-TN, MAP2, PSD-95, and GRIP1, but not the pre-synaptic marker synpatophysin (FIG. 7D). More specifically, in the showing of FIG. 7A, hippocampal slices were treated as indicated, and stained with Hoechst to outline the slices, and with anti-CD11b and anti-SRA to evaluate microglia activation. Aβ treatment caused increased staining of CD11b and SRA. A magnified image from an outlined SRA-immunoreactive area is shown on the far right to demonstrate the activated morphology of microglia. In FIG. 7B, NO production was measured as nitrite level in the conditioned medium as described in FIG. 2F and normalized to the amount of total protein of the slice. Treatment with AβO and LPS increased NO production from hippocampal slices (n=4, # p<0.001 compared with the “control” group). The AβO-induced increase was prevented by 20 μM doxycycline and 1 μM TRAM-34 (n=4, * p<0.05 compared with the “AβO” group). Doxycycline or TRAM-34 alone did not affect NO production. In FIG. 7C, paired consecutive slices received the same indicated treatment. One slice was then used for PI uptake study for neuronal damage, and the other for CD11b staining for activated microglia. There was low background PI uptake and CD11b staining. AβO significantly enhanced PI uptake and CD11b staining, which were ameliorated by doxycycline and TRAM-34. The locations of hippocampal subfields CA1, CA3 and dentate gyrus (DG) are indicated. FIG. 7D shows a Western blot analysis of lysates from hippocampal slices with indicated treatment, analyzed by antibodies to dendritic proteins Ac-TN and MAP2, postsynaptic proteins PSD95 and GRIP1, and the presynaptic protein synaptophysin. These data, as shown in FIGS. 7A through 7D, indicate that AβO activated microglia and caused indirect, microglia-mediated neurotoxicity in hippocampal slices. The treatment consisted of 20 nM AβO for 24 hrs.

NO is the Major Mediator of 40-Induced Microglial Neurotoxicity.

NO was found to be a major inflammatory mediator released by AβO-stimulated microglia, Applicants tested whether NO mediates neurotoxicity. Applicants used two selective inhibitors of iNOS, N-iminoethyl-Llysine (L-NIL) (57) and N-[(3-aminomethyl)benzyl]acetamidine (1400 W) and found that both compounds significantly blocked the increased NO release induced by AβO (FIG. 8A). When AβO-treated microglia were concurrently treated with iNOS inhibitors, their CM were no longer neurotoxic (FIG. 8B). When applied to cultured hippocampal slices 1 hr prior to AβO application, both iNOS inhibitors reduced NO release (FIG. 8C) and inhibited neuronal damage as indicated by reduced PI uptake (FIG. 8D). More specifically, in FIG. 8A, NO production was quantified as described in FIG. 2E. The AβO-induced increase was prevented by 5 μM 1400 W and 100 μM L-NIL (n=3, * p<0.01 compared with the “AβO” group). In FIG. 8B, AβO-induced microglial neurotoxicity was evaluated as described above with respect to FIG. 5. Control or AβO-treated microglia cultures were at the same time treated with vehicle, 1400 W, or L-NIL for 24 hrs. Hippocampal neuron cultures were then treated with 25% CM from each microglial culture. Neuronal viability was determined after 24 hrs. Both 1400 W and L-NIL blocked the AβO-induced microglial neurotoxicity (n=3, * p<0.001 compared with the “AβOCM/vehicle” group). In FIG. 8C, NO released by hippocampal slices was measured as described above with respect to FIG. 7B. Both 1400 W and L-NIL blocked AβO-induced NO production (n=3, * p<0.001 compared with the “AβO” group). In FIG. 8D, hippocampal slices were treated with indication conditions and the PI uptake assay was performed as described above with respect to FIG. 7C. The same slices were then immunostained with NeuN that marked neuronal nuclei (NeuN: green; PI uptake: red). A magnified image from an outlined area in the CA1 region of the A_O-treated hippocampal slice is shown on the right upper panel to demonstrate the substantial colocalization of NeuN-immunoreactivity and PI fluorescence. Both 1400 W and L-NIL blocked A_O-induced neuronal damage as indicated by significantly reduced PI uptake.

These effects were unlikely to be caused by direct actions of the compounds on neurons because neurons do not express KCa3.1 as discussed above, and doxycycline was shown to provide neuroprotection by regulating microglia, but not via neuronal mechanisms. Although a direct anti-amyloidogenic property of doxycyline was previously shown, the above-described experimental results do not indicate any direct effect on AβO because there was no effect of doxycycline on the morphology, size, or neurotoxic activities of AβO. Therefore, in the more physiological hippocampal slice cultures, low nanomolar AβO caused indirect neurotoxicity by activating microglia.

The experimental data summarized above show that Aβ is able to activate microglia at concentrations at least 10-fold lower than those used to induce direct neurotoxicity. Low nanomolar AβO activates microglia to release soluble neurotoxic factors and thus indirectly damages the integrity of neurons and synapses. AβO stimulates a unique neuroinflammatory pattern with increased NO generation but without the production of a regular panel of inflammatory mediators such as prostaglandin E2, glutamate, and the cytokines TNF-α, IL1-β, and IL6. These observations were reproduced using the more physiological hippocampal slices in addition to dissociated microglia cultures. Neurotoxicity was ameliorated by a well-studied inhibitor of microglia activation, doxycycline, and by the KCa3.1 blocker TRAM-34. Therefore, our results support the possibility that the early neurotoxicity seen during the initial buildup of AβO might be mediated, at least in part, by microglia.

Soluble AβO extracted from AD hippocampi were about 50 times more potent than synthetic AβO in activating microglia, further suggesting a role of AβO in activating microglia. The reason for the higher potency of brain-derived AβO is not known, but is possibly due to the presence of co-fractionated costimulators or in vivo modifications of brain-derived Aβ that are not present in synthetic Aβ peptides.

The ability of fAβ to activate microglia is generally low or absent when fAβ is the only stimulant; activation requires micromolar concentrations (2-100 μM) of fAβ and enhancement by costimulators such as γ-interferon, lipopolysaccharides, advanced glycation endproducts, complement factors, and MCSF. A few reports have showed that unaggregated forms of Aβ at micromolar concentrations were able to activate microglia. Freshly solubilized, non-aggregated Aβ-42 at 500 nM has been reported by others to activate microglia in the presence of CD40 ligand. It has also been reported that activation of microglia by variably prepared ADO, in concentrations ranging from 2 to 50 μM, was equally or less potent than fAβ in activating microglia. In contrast to previous reports, Applicants used AβO at concentrations about 1,000 times lower and were able to show AβO's significantly higher potency than that of fAβ or unaggregatd Aβ in activating microglia. Because this activity has a bell-shaped concentration-response curve, which maximizes at 20-50 nM Aβ, it was not recognized in prior studies using μM or high nM Aβ.

Applicants' data indicate that detrimental effects of AβO upon synapses were ameliorated by inhibitors of microglia activation, and therefore support an alternative, microglia-mediated mechanism of synaptic dysfunction. These data further show that AβO at low nM concentrations activates microglia and causes reduced levels of critical dendritic and postsynaptic proteins in both dissociated neuronal cultures and hippocampal slices. Applicants also found a pattern of preferential postsysynaptic damage mediated by AβO-activated microglia similar to those found in Tg2576 transgenic mice and in human AD cortex, suggesting a pathological relevance.

It has been determined that microglia processes make regular direct contact with synapses and that prolonged contact increases the turnover of synapses. Therefore Applicants hypothesize that synaptic AβO might attract microglia by a potent chemotactic effect and promote a synapse-centered neuroinflammatory reaction to damages synapses. Supporting this notion, a previous study showed that the inhibition of NMDA receptor-dependent long term potentiation by soluble Ap (500 nM, which might contain oligomers) can be prevented by minocycline, a microglia activation inhibitor in the same class as doxycycline, and iNOS inhibition to reduce NO production from microglia. NO is the only AβO-promoted neuroinflammatory mediator we have so far uncovered.

TRAM-34 is a small molecule that selectively blocks the intermediate-conductance calcium-activated potassium channel KCa3.1. The data described herein provide the first evidence that a specific K⁺ channel regulates Aβ-induced microglia activation and neurotoxicity. KCa3.1 regulates Ca²⁺-signaling by maintaining a negative membrane potential through K⁺ efflux, thus facilitating Ca²⁺ entry through CRAC (calcium-release activated Ca²⁺ channel), a channel responsible for the store-operated Ca²⁺ entry required for microglia activation. The anti-inflammatory and neuroprotective properties of KCa3.1 blockers have been shown in models of traumatic brain injury, multiple sclerosis, and retinal ganglion cell degeneration after optic nerve transection. In addition, TRAM-34, although inhibiting microglia-mediated neurotoxicity, does not affect the beneficial activities of microglia such as migration and phagocytosis. Accordingly, the present invention provides compositions and methods by which KCa3.1 blockers, targeting microglia selectively because of the microglia-restrictive cellular expression of KCa3.1 in the CNS, provide a novel anti-inflammatory effects in subjects suffering from or in the process of developing AD. Thus, inhibition or blockade of KCa3.1 constitutes a useful therapeutic method for reducing the detrimental effects of microglia-mediated neurotoxicity, such as in Alzheimer's disease, while preserving beneficial microglial effects.

Further in accordance with the present invention, inhibition of microglial activity by KCa3.I blockade in AD can preserve the beneficial functions of microglia such as phagocytosis of amyloid-beta deposits while inhibiting their deterimenatal effects like microglia mediated neuronal killing and the production on NO and inflammatory cytokines. This is additionally evidenced by the data shown graphically in FIG. 9. In these data, microglia were treated with 50 nM A130, which contained 75% unlabeled Aβ1-42 and 25% Aβ1-42 labeled with HiLyte Fluor488 (AnaSpec) for fluorescent detection. After 1 h incubation, Aβ uptake was determined by flow cytometry. Shown in the graph of FIG. 9 are means±S.E.M. from 3 independent experiments.

### Example 2

### Neuroprotection Following Ischemic, Annoxic or Hypoxic Event by Inhibition KCa3.1

Microglia and brain infiltrating macrophages significantly contribute to the secondary inflammatory damage in the wake of ischemic stroke. The following is an example, which demonstrates that inhibition of KCa3.1 (IKCal/KCNN4) reduces microglia and macrophage activation.

Using an HPLC/MS assay, Applicants first confirmed that our small molecule KCa3.1 blocker TRAM-34 effectively penetrates into the brain and achieves micromolar plasma and brain concentrations following intraperitoneal (i.p.) injection. Applicants then subjected male Wistar rats to 90 min of middle cerebral artery occlusion (MCAO) and administered either vehicle or TRAM-34 (10 or 40 mg/kg i.p. twice daily) for 7 days starting 12 h after reperfusion. Both compound doses reduced infarct area by ˜50% as determined by H&E staining on day-7 and the higher dose also significantly improved neurological deficit. Significant reductions in ED1⁺ activated microglia and TUNEL-positive neurons were observed as well as increases in NeuN⁺ neurons in the infarcted hemisphere. These findings suggest that KCa3.1 blockade constitutes an attractive approach for the treatment of ischemic stroke because it is still effective when initiated 12 hours after the insult.

In addition to directly causing neuronal damage, focal ischemic stroke elicits a strong and long-lasting inflammatory response. Activated by multiple stimuli, which include hypoxia, neuronal debris, ATP and glutamate, microglia retract their branched processes, round up and transform into “reactive” microglia. Partial breakdown of the blood-brain barrier additionally promotes the infiltration of macrophages, neutrophils and activated T cells from the blood. In both rodent models of cerebral ischemia and in histopathological studies on human postmortem brain sections activated microglia/macrophages are abundant in the infarcted area and the peri-infarct zone 18-96 hours after an ischemic insult, and are still present in chronic cystic stages months after a stroke. More recen PET imaging in ischemic stroke patients demonstrated microglia activation in the peri-infarct zone on a slightly more delayed time scale: starting at 72 hours and lasting for at least 4 weeks. While microglia can of course exert neuroprotective functions by releasing neurotrophic growth factors such as brain-derived neuroprotective factor (BDNF) or phagocytosing debris and potentially even invading neutrophils, activated microglia/macrophages are also the main source of inflammatory cytokines such as IL-1β and TNF-α, reactive oxygen species, nitric oxide and cyclooxygenase-2 reaction products.

TRAM-34 blocks the KCa3.1 channel with an IC₅₀ of 20 nM and exhibits 200-1500 fold selectivity over other IC channels. KCa3.1 is expressed on proliferating fibroblasts, dedifferentiated vascular smooth muscle cells, and on immune cells including microglia and macrophages, activated CCR7⁺ T cells and IgD B cells. In all these cells KCa3.1 is part of signaling cascades that involve relatively global and prolonged calcium rises during cellular proliferation, cytokine secretion and volume regulation. KCa3.1 channels are voltage-independent and only require a small increase in intracellular calcium to open and then maintain a negative membrane potential through IC efflux. KCa3.1 channels thus provide the driving force for store-operated inward-rectifier calcium channels like CRAC (calcium-release activated Ca²⁺ channel) or transient receptor potential channels like TRPC1.

Particularly in microglia, KCa3.1 has been shown to be involved in respiratory bursting, migration, proliferation and LPS or amyloid-β oligomer induced nitric oxide production as well as in microglia-mediated neuronal killing in cultures and organotypic hippocampal slices, suggesting that KCa3.1 suppression might be useful for reducing microglia activity in stroke, traumatic brain injury, multiple sclerosis and Alzheimer's disease. It has previously been reported that intraocular injection of the KCa3.1 blocker TRAM-34 reduced retinal ganglion cell degeneration after optic nerve transection in rats and that TRAM-34 treats experimental autoimmune encephalomyelitis (EAE) in mice. In the nerve transection study KCa3.1 blockade did not prevent microglia from aligning with damaged axons and from phagocytosing damaged neurons but increased the number of surviving retinal ganglion cells presumably by reducing the production and/or secretion of neurotoxic molecules in the retina. In addition, it has been reported that two structurally different KCa3.1 inhibitors, a triarylmethane and a cyclohexadiene, reduced infarct volume and brain edema following traumatic brain injury caused by acute subdural haematoma in rats.

Applicants have studied whether KCa3.1 blockade such as by TRAM-34, which inhibits LPS-stimulated p38 mitogen-activated protein kinase (MAPK) activation but not nuclear-factor κB (NF-κB) activation in microglia, might preferentially target microglia activities that are involved in neuronal killing without affecting beneficial functions such as scavenging of debris.

### A. Materials and Methods

MCAO with 7 Days of Reperfusion

Adult male Wistar rats weighing 160-180 g were purchased from Charles River (Wilmington, Mass.), acclimatized to the new vivarium for 5-7 days and used for the surgery when they weighed 200-230 g. Rats were anesthetized using box induction with 5% isoflurane and then maintained on 0.5%-1.5% isoflurane in medical grade oxygen via a facemask. In order to assure consistent reduction of cerebral blood flow (CBF) throughout the procedure, we affixed a small hand-made adaptor for the Laser Doppler probe (Moor Instruments, Wilmington, Del.) to the surface of the skull. The center of the adapter was 5 mm lateral to the central fusion line and 2.5 mm posterior to bregma. Instant adhesive and dental cement were applied to the base and around the edges of the small plastic adapter to hold the Doppler probe. The adapter with the attached probe remained in place throughout the MCAO surgery to confirm continuous occlusion and later the establishment of reperfusion. Focal cerebral ischemia was then induced by occlusion of the left middle cerebral artery (MCA). The left common carotid artery was surgically exposed, the external carotid artery was ligated distally from the common carotid artery, and a silicone rubber coated nylon monofilament with a tip diameter of 0.43±0.02 mm (Doccol Corp., Redlands, Calif.) was inserted into the external carotid artery and advanced into the internal carotid artery to block the origin of the middle cerebral artery (when maximum CBF reduction observed). The filament was kept in place for 90 min and then withdrawn and removed from the blood vessel to restore blood supply. Rats received TRAM-34 at 10 mg/kg, 40 mg/kg or vehicle (Miglyol 812 neutral oil at 1 μl/g) twice daily i.p. for 7 days starting 12 hour after reperfusion. Neurological deficits were scored according to a 4-score test and a tactile and/or proprioceptive limb-placing test as follows: 1) 4-score test (higher score for more severe neurological deficits): 0=no apparent deficit; 1=contralateral forelimb is consistently flexed during suspension by holding the tail; 2=decreasing grip ability on the contralateral forelimb while tail pulled; 3=spontaneous movement in all directions but circling to contralateral side when pulled by the tail; 4=spontaneous contralateral circling or depressed level of consciousness. 2) 14-score limb placing test (lower score for more severe neurological deficits): Proprioception, forward extension, lateral abduction, and adduction were tested with vision or tactile stimuli. For visual limb placing, rats were held and slowly moved forward or lateral toward the top of a table. Normal rats placed both forepaws on the tabletop. Tactile forward and lateral limb placing were tested by lightly contacting the table edge with the dorsal or lateral surface of a rat's paw while avoiding whisker contact and covering the eyes to avoid vision. For proprioceptive hind limb placing, each rat was pushed along the edge of an elevated platform in order to test proprioceptive hind limb adduction. The paw was pulled down and away from the platform edge, and the ability to retrieve and place the paw on the table surface upon sudden release was assessed. For each test, limb placing scores were 0=no placing; 1=incomplete and/or delayed (>2 seconds) placing; or 2=immediate and complete placing. For each body side, the maximum summed visual limb placing score was 4 and the maximum summed tactile and proprioceptive limb placing score, including the platform test, was 10.

### Pharmacokinetics, Brain Concentrations and Plasma Protein Binding of TRAM-34

TRAM-34 was synthesized in our laboratory as previously described and its chemical identity and purity checked by ¹H-NMR and HPLC/MS. For intravenous application TRAM-34 was dissolved at 5 mg/ml in a mixture of 25% Cremophor®EL (Sigma-Aldrich, St. Louis, Mo.) and 75% PBS and then injected at 10 mg/kg into the tail vein of male Wistar rats. At various time points following the injection approximately 100-200 μl of blood were collected from a tail nick into EDTA blood sample collection tubes. For simultaneous determinations of plasma and brain concentrations TRAM-34 was dissolved in Miglyol 812 neutral oil (caprylic/capric triglyceride; Tradename Neobee M5®, Spectrum Chemicals, Gardena, Calif.) at 10 or 40 mg/ml and injected i.p. at 10 or 40 mg/kg. Blood samples were taken by cardiac puncture under deep isoflurane anesthesia. The right atrium was then cut open and 20 mL of saline slowly injected into the left ventricle to flush the blood out of the circulation. The rats were then sacrificed and brains removed. Plasma was separated by centrifugation and samples stored at −80° C. pending analysis. Plasma and homogenized brain samples were purified using C18 solid phase extraction (SPE) cartridges. Elutioned fractions corresponding to TRAM-34 were dried under nitrogen and reconstituted in acetonitrile. LC/MS analysis was performed with a Hewlett-Packard 1100 series HPLC stack equipped with a Merck KGaA RT 250-4 LiChrosorb RP-18 column interfaced to a Finnigan LCQ Classic MS. The mobile phase consisted of acetonitrile and water, both containing 0.2% formic acid. With a flow rate of 1.0 ml per min the gradient was ramped from 20/80 to 70/30 in 5 min, then to 80/20 over 11 min, to 5/95 till 16.5 min and finally back to 80/20 till 38 min. With the column temperature maintained at 30° C., TRAM-34 eluted at 14.4 min and was detected by a variable wavelength detector (VWD) set to 190 nm and the MS in series. Using electrospray ionization/ion trap MS (capillary temperature 270° C., capillary voltage 1V, tube lens offset −15 V, positive ion mode) TRAM-34 was quantified by its base peak of 277 m/z (2-chlorotrityl fragment) and concentrations calculated with a 5-point calibration curve from 25 nM to 2.5 Concentrations above 2.5 μM were quantified by their UV absorption at 190 nm. The related compound TRAM-46 (base peak of 261 m/z, 2-fluorotrityl fragment) was used as an internal standard.

The percentage of plasma protein binding for TRAM-34 was determined by ultrafiltration. Rat plasma was spiked with 50 and 100 μM TRAM-34 in 1% DMSO and the sample loaded onto a Microcon YM-100 Centrifugal Filter (Millipore Corporation, Bedford, Mass.) and centrifuged at 14000 rpm for 15 min at RT. The centrifugate (=free TRAM-34) was directly analyzed for TRAM-34 by HPLC-MS. The retentate was collected by inverting the filter into an Eppendorf tube and spinning at 14000 rpm for 15 min. The retentate then underwent sample preparation as per the above-described procedure for determining total TRAM-34 concentration in plasma. The plasma protein binding of TRAM-34 was found to be 98±0.5% (n=3) and the unbound (=free) fraction 2.0±0.4%.

### Assessment of Infarct Area

Rats were euthanized with an overdose of isoflurane. Blood samples for determination of electrolytes, pH, pCO₂, glucose and hemoglobin (I-STAT; Abbott, Princeton, N.J.) were drawn from the vena cava and brains quickly removed and sectioned into eight 2-mm thick slices starting from the frontal pole. Slices were then fixed in 10% buffer formalin embedded in paraffin and sectioned at 5 Sections were stained with hematoxylin & eosin and scanned. The resulting jpg images were analyzed in Adobe Photoshop CS3 for infarct area using the Magnetic Lasso tool to outline the area and the Histogram tool to determine the number of pixels in the respective area. Percent infarct for each slice was calculated as: (pixels in ipsilateral side/pixels in whole control hemisphere)×100. Percentage of total infarct area of whole hemisphere was calculated as: (summation of pixels in infarct from 8 slices/summation of pixels in whole control hemisphere from 8 slices)×100. The degree of brain shrinkage was calculated from the same data.

### Immunohistochemistry

Sections were dewaxed with xylene, rehydrated through an alcohol gradient, and heated with 10 mM Na citrate (pH 6) in a microwave for 15 min to retrieve antigenic determinants After treatment with 1% H₂O₂ to inactivate endogenous peroxidase activity and blocking with 5% goat serum in PBS, the sections were incubated overnight at 4° C. with the primary antibody in PBS/2% goat serum. The following primary antibodies were used: KCa3.1 (1:500; AV35098, Sigma), CD68 (ED1, 1:1000; Serotec Raleigh, N.C.), and NeuN (1:1000; A60, Millipore, Calif.). The polyclonal anti-KCa3.1 antibody, which recognizes human, rat and mouse KCa3.1 was tested for specificity with spleen and vascular sections from KCa3.1-wild-type and KCa3.1^{-/-} mice. Bound primary antibodies were detected with a biotinylated donkey anti-mouse IgG secondary antibody for CD68 and NeuN, or with a biotinylated goat anti-rabbit IgG secondary antibody (both 1:500, Jackson ImmunoResearch, West Grove, Pa.) for KCa3.1 followed by a horseradish peroxidase-conjugated avidin complex (Vectastain Elite ABC Kit, Vector Laboratories, Burlingame, Calif.). Peroxidase activity was visualized with 3,3′-diaminobenzidine (DAB Substrate Kit for Peroxidase, Vector Laboratories). Sections were counterstained with hematoxylin (Fisher, Pittsburg, Pa.), dehydrated and mounted with Permount (Fisher, Pittsburg, Pa.). AβOptosis was assessed with the AβOpTag Peroxidase in Situ AβOptosis detection kit (Millipore, Billerica, Mass.) according to the manufacturer's protocol.

ED1 stains CD68, a lysosomal membrane protein, which is mainly found in phagocytosing macrophages and reactive microglia. At 1:1000 the antibody produced no stain on resting microglia. Infiltration of ED1⁺ cells was evaluated. Sections stained for ED1 were photographed and the resulting photos composited into whole-slide images with Photoshop. The Magnetic Lasso tool was used to outline hemisphere borders, brown pixels were selected with the magic wand tool and the number of brown pixels determined with the Histogram tool. The results are reported as brown=ED1⁺ positive pixels per one millimeter square area (pixels/mm²). NeuN is a DNA-binding, neuron-specific protein present in neuronal nuclei, perikarya and some proximal neuronal processes. Strong nuclear staining suggests proper nuclear regulatory protein function representative of a healthy neuron. Sections stained for NeuN were photographed and the resulting photos composited into whole-slide images with Photoshop. NeuN and TUNEL positive cells in the infracted hemisphere were counted with the Photoshop CS3 extended count tool.

### Statistical Analysis

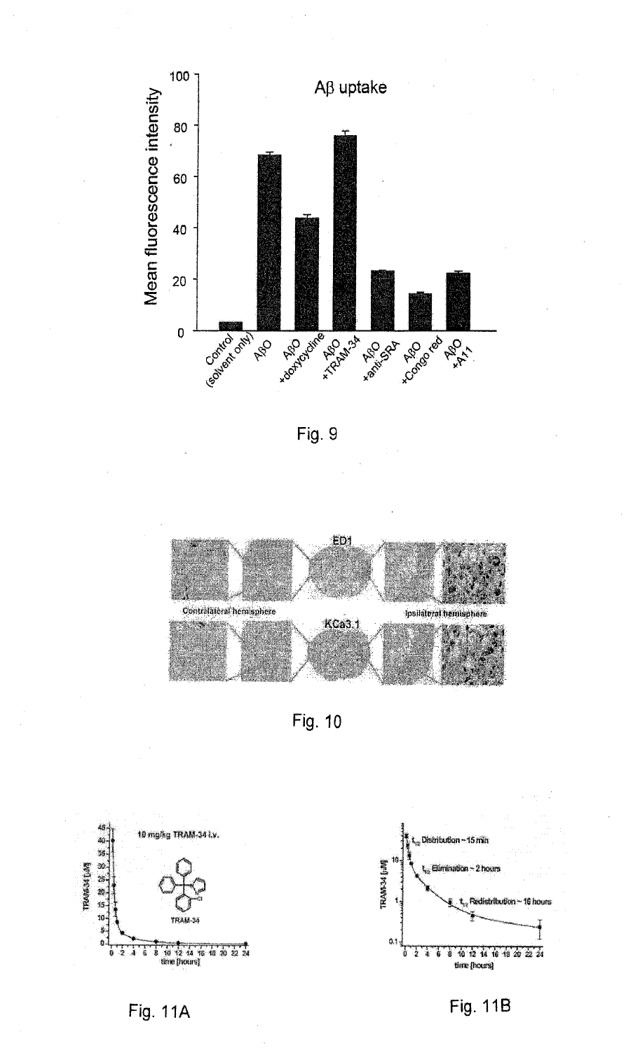

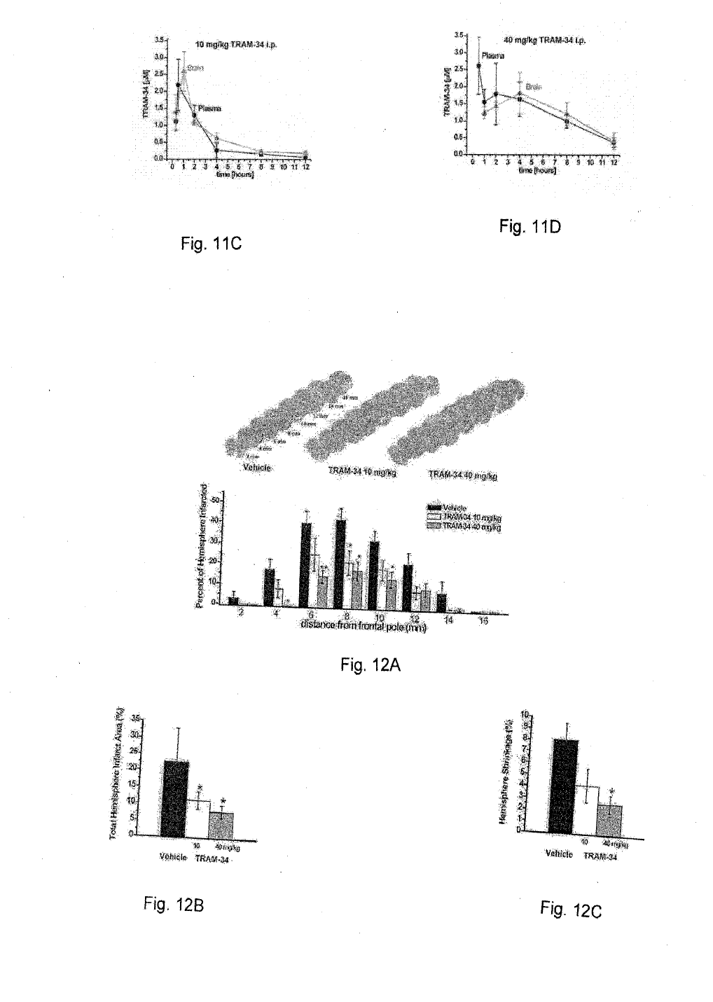

Statistical analyses of infarct area, neurological deficit scoring and IHC were performed with one-way analysis of variance (ANOVA; Origin software) followed by post-hoc pair-wise comparison of the different groups using Tukey's method, also referred to as honestly significant difference test, as recommended by Schlattmann and Dirnagle for MCAO studies. P<0.05 was used as the level of significance. *=P<0.05, **=P<0.01, ***=P<0.001. A11 data with the exception of the pharmacokinetic data in FIG. 11, which shows mean±SD, are given as mean±SEM.

### B. Results

MCAO with 7 Days of Reperfusion Induces Substantial Activation of Microglia/Macrophages Expressing KCa3.1

In determining the optimal length of reperfusion for this experiment, it was noted that 48 hours of reperfusion only induced mild to moderate inflammation as measured by the number of ED1⁺ (=CD68⁺) activated microglia/macrophages (data not shown), while seven days of reperfusion resulted in a dramatic increase in ED1⁺ cells in the infarcted brain areas as shown in FIG. 10, which depicts paraffin-embedded sections from a 90 min MCAO with 7 days of reperfusion for ED1 and KCa3.1. FIG. 10 shows that activated microglia/macrophages in infarcted brain areas express KCa3.1.

Staining of serial sections with a polyclonal anti-KCa3.1 antibody, which did not produce any stain on lymphoid and vascular tissues from KCa3.1^{-/-} mice, revealed strong KCa3.1 expression on cells with the round or “ruffled” shape characteristic of reactive microglia/macrophages in the infarcted areas. While the two stains clearly were on the same cells, they did not strictly co-localize because CD68 is a lysosomal protein, whereas the KCa3.1 channel is expressed on the plasma membrane and traffics through the ER. As seen on the microvessel in the right panels of FIG. 10, KCa3.1 protein was also detectable on vascular endothelial cells in keeping with the known expression of KCa3.1 in vascular endothelium and its role in the endothelium-derived hyperpolarizing-factor (EDHF) response. The abundantly present KCa3.1⁺ microglia in the infarcted area suggest that KCa3.1 inhibition might be of therapeutic benefit for curbing the secondary inflammatory damage in the wake of ischemic stroke.

### Pharmacokinetics and Brain Permeability of TRAM-34

To reduce brain inflammation the KCa3.1 blocker TRAM-34 should ideally reach pharmacologically active concentrations in the brain. In order to address this question and to determine TRAM-34's pharmacokinetics in rats we established an HPLC/MS assay to measure TRAM-34 concentrations in plasma and tissue. Following intravenous administration at 10 mg/kg, total TRAM-34 plasma concentrations fell from a peak of 40 μM at 8 min after application to 250 nM at 24 hours. This decay in plasma levels was best fitted tri-exponentially reflecting a 3-compartment model with rapid distribution from blood into tissue followed by elimination and slow repartitioning from body fat acting as a deep compartment back into plasma (FIG. 11A, 11B). The half-life of TRAM-34 was calculated from the elimination part of the plot and found to be ˜2 hours, which is slightly longer than the 1-hour half-life we previously determined in mice. We next injected TRAM-34 intraperitoneally at 10 and 40 mg/kg and measured total plasma and brain concentrations at various time points (FIG. 11C, 11D). Following administration of 10 mg/kg total plasma and brain levels of TRAM-34 initially peaked around 2.5 between 30 min and 1 hour of application and then rapidly fell to 58±9 nM in plasma and 191±41 nM in homogenized brain tissue within 12 hours. The higher TRAM-34 dose of 40 mg/kg in contrast resulted in a much more protracted absorption from the intraperitoneal space (FIG. 11D) and achieved plasma and brain concentrations exceeding 1 μM for 8 hours and only slowly falling to roughly 400 nM at 12 hours. Since TRAM-34 had been previously administered subcutaneously once daily to prevent restenosis following angioplasty in rats we also determined TRAM-34 plasma levels after s.c. injection. In comparison to i.v. or i.p. application, TRAM-34 showed very poor bioavailibility following s.c. administration and we needed to use 120 mg/kg to achieve plasma peaks of 2.5±1 μM (data not shown). Release was further slow and varied greatly in its kinetics between individual animals, making subcutaneous application unsuitable for short-term in vivo trials despite the obvious convenience of once daily application of a high dose. In order to determine how much of the total TRAM-34 concentration was free and thus available for blocking KCa3.1, we also determined TRAM-34's plasma protein binding by ultrafiltration. In keeping with TRAM-34's high lipophilicity, plasma protein binding was high (98%) leaving a free concentration of only 2%. More specifically, FIGS. 11A through 11D show in graphic form the pharmacokinetics of TRAM-34 in rats. FIG. 11A shows total TRAM-34 plasma concentrations in rats (n=3) following i.v. administration at 10 mg/kg. The data are best fitted as triexponential decay in keeping with a three compartment model. Inset: Structure of TRAM-34. FIG. 11B contains the same data as in FIG. 11A shown on a logarithmic scale to better visualize the three slopes. Total TRAM-34 plasma () and brain (▴, density assumed as 1 g/mL) concentrations following i.p. injection at 10 mg/kg (C) or 40 mg/kg (D). Each data point in (C) and (D) is from three animals. A11 values are mean±SD.

Taken together these results demonstrate that TRAM-34 has reasonably good pharmacokinetics in rats and effectively reaches the brain even when the blood-brain barrier is intact (C_{brain}/Cₚₗₐₛₘₐ=1.2). Based on its plasma half-life of 2 hours and its fast availability following intraperitoneal application we decided to administer TRAM-34 twice daily intraperitoneally at 12 hour intervals.

KCa3.1 Blockade with TRAM-34 Reduces Infarction and Microglia Activation in MCAO with 7 Days of Reperfusion when Treatment is Started 2 Hours after Reperfusion

In preliminary experiments we induced relatively mild infarcts by reducing cerebral blood flow by only 50% (control: 51.2±8.2% flux reduction, mean±SD, n=10; TRAM-34: 49.0±7.4% flux reduction, n=11) and then administering TRAM-34 at 10 mg/kg twice daily for 7 days starting 2 h after reperfusion. Under these conditions we found a reduction in mean infarct area from 18.8±3.5% of the ipsolateral triphenyltetrazolium chloride positive hemisphere area (n=10) in controls to 6.6±1.9% in TRAM-34 treated animals (n=11; mean±SEM, P=0.007). As shown in FIGS. 11A-11D, an analysis was made of each affected brain slice from 2 mm to 16 mm from the frontal pole of the brain and a plot of the mean infarct area in the two groups. In order to access if the reduction in infarct area by TRAM-34 was accompanied by a reduction in microglia/macrophage activation, we stained formalin fixed sections from the center of the infarct in the 8 and 10-mm slices from 5 animals of each group and evaluated ED1⁺ staining according to Lehr et al. This pixel based method is useable for evaluating the large numbers of the intensely ED1⁺ stained and irregularly shaped microglia/macrophages. TRAM-34 treatment reduced the amount of activated microglia/macrophages in the infarcted hemisphere (n=5 per group).

KCa3.1 Blockade with TRAM-34 Reduces Infarction in MCAO with 7 Days of Reperfusion when Treatment is Started 12 Hours after Reperfusion

The above-described results suggest that KCa3.1 blockade can indeed reduce infarction and microglia activation. In view of this, Applicants perfoiined a second set of MCAO experiments where cerebral blood flow was reduced more severely and evaluated hemotoxylin & eosin (H&E) defined lesion area since this method is known to be more accurate for aged infarcts than TTC. However, in order to be able to prepare undamaged μm thin sections from the often brittle and delicate aged infarcts, the 2-mm coronal slices were fixed in 10% formalin for 1 day and the resulting paraffin blocks trimmed until several undamaged section could be obtained from each slice. In order to better simulate possible treatment conditions in the clinic we also delayed the start of the TRAM-34 treatment until 12 hours after reperfusion.

As in our previous experiments, male Wistar rats were subjected to 90 min of MCAO with 7 days of reperfusion and then treated with either TRAM-34 at 10 mg/kg or 40 mg/kg or vehicle twice daily starting 12 hour after successful reperfusion. Cerebral blood flow reduction was 67.3±9.6% in the controls (n=8), 71.4±5.8% in the high-dose TRAM-34 group (n=8) and 67.9±5.9% in the low-dose TRAM-34 group (n=6; values are means±SD), which also depicts a representative example of a laser Doppler recording during MCAO surgery and reperfusion.

TABLE 1
Physiological Parameters

| | | MCAO + | MCAO + |
|---|---|---|---|
| | MCAO + | TRAM-34 | TRAM-34 |
| | vehicle | (10 mg/kg) | (40 mg/kg) |
| After Reperfusion | | | |
| Na⁺ (mmol/L) | 133.2 ± 3.0 | 134.4 ± 2.3 | 135.5 ± 2.3 |
| K⁺ (mmol/L) | 6.4 ± 1.7 | 6.3 ± 1.1 | 5.7 ± 0.5 |
| Cl⁻ (mmol/L) | 97.4 ± 1.9 | 99.0 ± 2.9 | 99.7 ± 4.0 |
| pH | 7.29 ± 0.13 | 7.34 ± 0.1 | 7.4 ± 0.13 |
| pCO₂ (mm Hg) | 78.9 ± 27.8 | 66.6 ± 28.3 | 60.8 ± 33.4 |
| HCO₃⁻ (mmol/L) | 36.0 ± 4.1 | 33.8 ± 6.4 | 32.5 ± 5.7 |
| BUN (mg/dL) | 22.4 ± 6.2 | 12.8 ± 7.2 | 22.2 ± 4.0 |
| Glucose (mg/dL) | 203.4 ± 53.2 | 179.2 ± 48.2 | 171.8 ±54.3 |
| Hemoglobin (g/dL) | 13.0 ± 0.6 | 14.3 ± 1.5 | 14.5 ± 1.4 |
| Day-7 after MCAO | | | |
| Na⁺ (mmol/L) | 136.9 ± 2.3 | 137.7 ± 4.1 | 138.8 ± 3.5 |
| K⁺ (mmol/L) | 4.6 ± 0.7 | 4.8 ± 1.1 | 4.2 ± 0.3 |
| Cl⁻ (mmol/L) | 102.3 ± 0.8 | 103.8 ± 4.3 | 103.4 ± 2.4 |
| pH | 7.4 ± 0.06 | 7.3 ± 0.06 | 7.4 ± 0.07 |
| pCO₂ (mm Hg) | 53.8 ± 6.1 | 58.1 ± 12.7 | 49.3 ± 7.3 |
| HCO₃⁻ (mmol/L) | 30.1 ± 0.4 | 29.7 ± 2.5 | 29.2 ± 1.3 |
| BUN (mg/dL) | 14.3 ± 1.6 | 18.7 ± 6.8 | 19.6 ± 4.7 |
| Glucose (mg/dL) | 200.3 ± 29.5 | 185.2 ± 41.2 | 201.1 ± 40.1 |
| Hemoglobin (g/dL) | 12.4 ± 0.9 | 12.7 ± 2.1 | 13.3 ± 1.5 |

Physiological parameters (±SD) measured by I-STAT in venous blood samples drawn after reperfusion from the tail vein or at sacrifice on day-7 after MCAO surgery from the vena cava. No significant differences were found between MCAO + vehicle (n = 8), MCAO + TRAM-34 (10 mg/kg; n = 6), and MCAO + TRAM-34 (40 mg/kg; n = 8). PCO₂ was elevated in all groups due to respiratory depression by isoflurane anesthesia. PCO₂ in tail vein samples from awake male rats is 49.9 ± 3.9 mm Hg (n = 5); 20 min of isoflurane anesthesia was observed to raise PCO₂ in tail vein samples to 57 ± 6.7 mm Hg (n = 5).

Table 1, above, shows that there were no differences with respect to plasma concentrations of Na⁺, K⁺, Cl⁻, HCO₃⁻, glucose, blood urea nitrogen, hemoglobin, pH, PCO₂ or hematocrit in venous blood samples taken after the surgery and at the time of sacrifice between animals subjected to 90 min of MCAO and treated with either vehicle or TRAM-34. (Please note that PCO₂ directly after surgery was elevated in all groups due to the respiratory depression from ˜2 h of isoflurane anesthesia). We also measured TRAM-34 concentration at the time of sacrifice, which was 12 hours after the last application, and found an average plasma concentration of 91±46 nM (n=6) for the 10 mg/kg group and 662±416 nM (n=7; mean±SD) for the 40 mg/kg group, while no TRAM-34 was detectable in vehicle treated animals. Treatment with TRAM-34 resulted in a significant reduction in H&E defined lesion area with the mean infarct size (FIG. 12B) being reduced from 22.6±3.6% in the controls (n=8) to 11.3±2.8% in rats treated with 10 mg/kg TRAM-34 (n=6, mean±SEM, P=0.039) and to 8.1±1.9% in rats treated with 40 mg/kg TRAM-34 (n=8; P=0.004). The treatment also tended to reduce brain shrinkage (FIG. 12C). However, the results were only statistically significant with 40 mg/kg TRAM-34 (P=0.013) but not for the 10 mg/kg group (P=0.11). More specifically, FIGS. 12A-12C show the effect of TRAM-34 on infarct area in rats subjected to 90 min of MCAO with 7 days of reperfusion. FIG. 12A shows H&E defined lesion areas in brain slices from 2 to 16 mm from the frontal pole from rats treated for 7 days starting 12 hours after reperfusion with vehicle (n=8), 10 mg/kg TRAM-34 (n=6) or 40 mg/kg (n=8). The graph of FIG. 12B shows the total hemisphere infarct area in the three groups and the graph of FIG. 12C shows the percentage of hemisphere shrinkage. A11 values are mean±SD.

KCa3.1 Blockade with TRAM-34 Reduces Neurological Deficit, Microglia Activation and Neuronal Death

Using both a 4-score neurological evaluation scale shown to correlate well with infarct sizes in the frontoparietal cortex and a 14-score tactile and proprioceptive limb-placing test rats were evaluated for neurological deficit 12 h after MCAO and then every 24 h for 7 days. The combination of both tests was chosen since filament MCAO in rats not only induces infarction in the major MCA territory, the lateral and parietal cortex, but also in the underlying striatum. Rats subjected to MCAO exhibited an average deficit score of 3 in the 4-score system (FIG. 13A) and a score of 4 in the tactile and proprioceptive 14-score system (FIG. 13B) 12 h after MCAO. These scores slowly improved in vehicle treated animals to an average of 2 in the 4-score and an average of 8 in the 14-score system by post-surgery day-7 probably reflecting the resolution of edema and partial compensation. (Please note that a normal rat has a score of 0 in the 4-score and a score of 14 in the 14-score system.) TRAM-34 treatment with 40 mg/kg commenced at 12 h after reperfusion started to significantly improve neurological deficit in both the 4-score and the 14-score system from day-5 or day-4 on and on day-7 treated rats displayed a score of 0.5 in the 4-score and of 12 in the 14-score system (FIGS. 13A and 13B). The lower TRAM-34 dose of 10 mg/kg only significantly improved neurological deficit in the more grade 14-score system and despite showing a positive trend towards improvement failed to significantly reduce deficit in the 4-score system. Interestingly, the deficit that high dose TRAM-34 treated animals consistently failed to recover was forelimb placement without vision in keeping with the fact that their infarcts were mostly restricted to the striatum. Vehicle treated animals in contrast exhibited both fore limb and hind limb impairment on day-7 indicating infarction in both the frontoparietal cortex and the striatum. More specifically, FIGS. 13A-13B illustrate the effect of TRAM-34 on neurological deficit. FIG. 13A shows neurological deficit in the 4-score system (normal rat=0). Scores at 12 hours after reperfusion are 3.1±0.20 in vehicle treated animals (n=8), 3.3±0.50 in the 10 mg/kg TRAM-34 group (n=6), and 3.0±0.80 in the 40 mg/kg TRAM-34 group (n=8, P=0.98) and are not significantly different between the groups. Scores at 196 h (=7 days) after reperfusion are 1.5±0.30 in vehicle treated animals n=8), 0.8±0.10 in the 10 mg/kg TRAM-34 group (n=6, P=0.24), and 0.4±0.01 in the 40 mg/kg TRAM-34 group (n=8, P=0.02). (B) Neurological deficit in the 14-score system (normal rat=14). Scores at 12 h after reperfusion are 4.3±0.94 in vehicle treated animals, 4.8±0.63 in the 10 mg/kg TRAM-34 group, and 3.4±0.46 in the 40 mg/kg TRAM-34 group and are not significantly different between the groups. Scores at 196 h (=7 days) after reperfusion are 7.5±0.46 in vehicle treated animals, 9.8±0.01 in the 10 mg/kg TRAM-34 group (P=0.004), and 11±0.38 in the 40 mg/kg TRAM-34 group (P<0.001). A11 values are mean±SD.

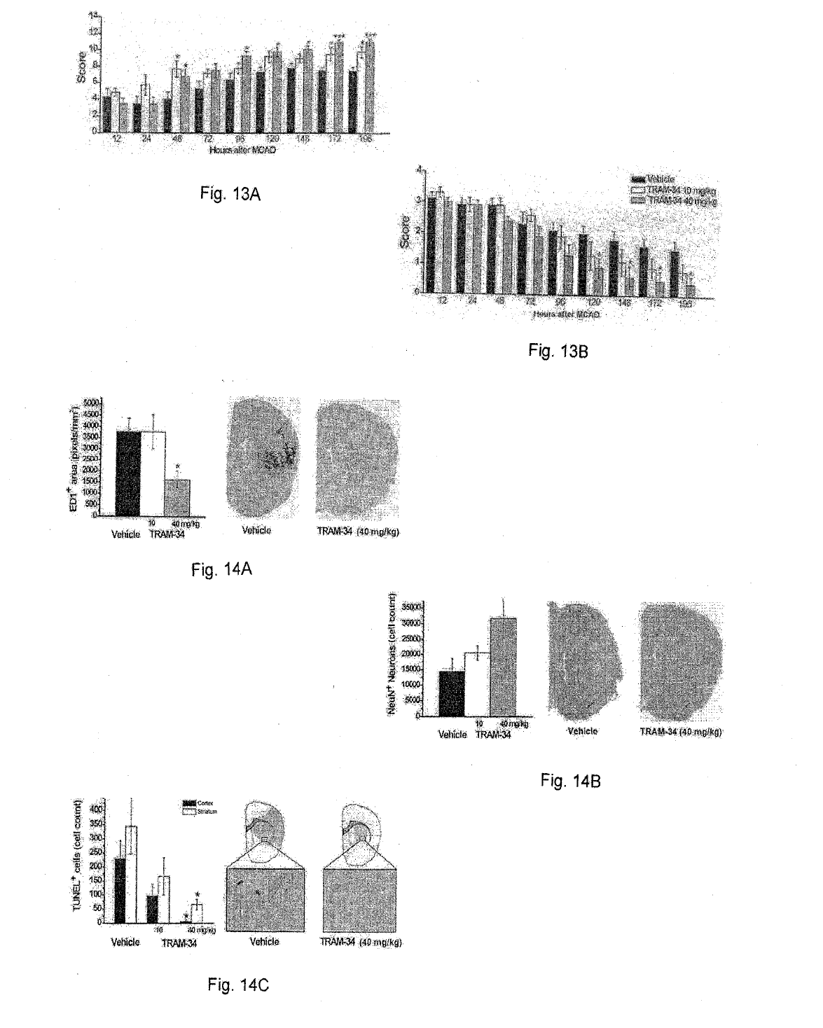

In order to determine if the delayed TRAM-34 application (12 hours after MCAO) also reduced microglia/macrophage activation similar to what we had previously seen when treatment was started 2 hours after reperfusion, we stained sections from the center of the infarct in the 8 and 10-mm slices from all animals in the vehicle, low-dose and high-dose TRAM-34 group for ED1⁺ microglia and determined the ED1⁺ area according to the pixel based method by Lehr et al. While the delayed administration of 10 mg/kg TRAM-34 did not result in a significant reduction in microglia activation, the higher TRAM-34 dose of 40 mg/kg reduced the ED1⁺ area from 3770.6±594.2 pixels/mm² to 1632.6±363.75 pixels/mm² (P=0.03) in the infracted hemisphere (FIG. 12). This reduction in microglia activation in brains from rats treated with 40 mg/kg TRAM-34 was accompanied by an increase in the average number of surviving NeuN⁺ neurons (P=0.052) in the infarcted hemisphere from sections from coronal slices 8 and 10 of all animals. As seen in FIGS. 14A-14C, an analysis of TUNEL positive cells in both the cortex and striatum further revealed that the 40 mg/kg dose of TRAM-34 significantly reduced the number of apoptotic cells in both locations (P=0.0067 for cortex and 0.033 for striatum). The lower dose, while showing a trend towards reducing the number of TUNEL-positive cells did not reach statistical significance (P=0.143 for cortex and 0.230 for striatum). More specifically, FIGS. 14 A through 14C show the effect of TRAM-34 on microglia activation and neuronal survival 7 days after MCAO. FIG. 14A shows ED1+ area (pixels/mm²) in the infarcted hemisphere from the 8- and 10-mm slices from all vehicle and TRAM-34 treated animals. FIG. 14B shows the number of surviving NeuN+ neurons in the infarcted hemisphere from the 8- and 10-mm slices from all vehicle and TRAM-34 treated animals. FIG. 14C shows the number of apoptotic TUNEL+ cells in the cortex and striatum in the infarcted hemisphere from all vehicle and TRAM-34 treated animals. A11 values are mean±SEM.

The calcium-activated K⁺ channel KCa3.1 plays an important role in several microglia functions such as respiratory burst, migration and microglia-mediated neuronal killing in vitro and in vivo. TRAM-34 is lipophilic and effectively crosses the blood-brain barrier. Based on the data provided in this example, KCa3.1 has a neuroprotective effect and reduces infarct in the rat ischemic stroke model tested. It is reasonable to conclude from these data that TRAM 34, when administered in doses effective to inhibit the calcium-activated K⁺ channel KCa3.1 in microglial cells will deter neuronal damage following ischemic, anoxic or hypoxic brain insult.

It is to be appreciated that the invention has been described hereabove with reference to certain examples or embodiments of the invention but that various additions, deletions, alterations and modifications may be made to those examples and embodiments without departing from the intended spirit and scope of the invention. For example, any element or attribute of one embodiment or example may be incorporated into or used with another embodiment or example, unless otherwise specified of if to do so would render the embodiment or example unsuitable for its intended use. Also, where the steps of a method or process have been described or listed in a particular order, the order of such steps may be changed unless otherwise specified or unless doing so would render the method or process unworkable for its intended purpose. A11 reasonable additions, deletions, modifications and alterations are to be considered equivalents of the described examples and embodiments and are to be included within the scope of the following claims.

What is claimed is:

1. A method for deterring microglia-mediated neurotoxicity in a human or non-human animal subject, said method comprising the step of inhibiting or blocking the intermediate-conductance calcium-activated potassium channel KCa3.1 in microglia.

2. A method according to claim 1 wherein the step of inhibiting or blocking the intermediate-conductance calcium-activated potassium channel KCa3.1 comprises administering to the subject a therapeutically effective amount of a substance that inhibits or blocks the KCa3.1 channel.

3. A method according to claim 2 wherein the substance comprises 1-[(2-chlorophenyl)diphenylmethyl]-1H-pyrazole (TRAM-34).

4. A method according to claim 1 wherein the inhibition or blockade of the intermediate-conductance calcium-activated potassium channel KCa3.1 reduces neurotoxic effects of the microglia without preventing beneficial effects of the microglia.

5. A method according to claim 4 wherein the subject has Aβ deposits and wherein the inhibition or blockade of the intermediate conductance calcium-activated potassium channel KCa3.1 reduces at least one neurotoxic effect of microglia selected from a) microglia-mediated neuronal killing, b) microglial production of NO and c) microglial cytokine production while not preventing microglia from phagocytosing Aβ deposits.

6. A method according to claim 1 wherein the method is performed to reduce neural damage in a subject suffering from a neurodegenerative disease.

7. A method according to claim 6 wherein the neurodegenerative disease is Alzeheimer's Disease.

8. A method according to claim 1 wherein the method is performed to reduce neural damage in a subject who has suffered or is suffering an ischemic, anoxic or hypoxic insult.

9. A method according to claim 8 wherein the ischemic, anoxic or hypoxic insult is due to at least one cause selected from a) ischemic stroke, b) hemorrhagic stroke, c) cardiac arrest and resuscitation, d) carbon monoxide poisoning, e) trauma, f) asphyxiation, g) strangulation, h) drowning, i) hemorrhagic shock, j) inhalant substance abuse or huffing, k) brain edema and 1) iatrogenic disruption of cerebral circulation during a surgery or other medical procedure.

10. The use of 1-[(2-chlorophenyl)diphenylmethyl]-1H-pyrazole (TRAM-34) in the manufacture of a pharmaceutical preparation for deterring microglia-mediated neurotoxicity in a human or non-human animal subject by inhibiting or blocking the intermediate-conductance calcium-activated potassium channel KCa3.1 in microglia.

11. A use according to claim 10 wherein the pharmaceutical preparation is for reducing neurotoxic effects of microglia without preventing beneficial effects of microglia.

12. A use according to claim 11 wherein the pharmaceutical preparation is for administration to a subject who has Aβ deposits and wherein the inhibition or blockade of the intermediate conductance calcium-activated potassium channel KCa3.1 reduces at least one neurotoxic effect of microglia selected from a) microglia-mediated neuronal killing, b) microglial production of NO and c) microglial cytokine production while not preventing microglia from phagocytosing Aβ deposits.

13. A use according to claim 10 wherein the pharmaceutical preparation is for reducing neural damage in subjects suffering from a neurodegenerative disease.

14. A use according to claim 10 wherein the pharmaceutical preparation is for reducing neural damage in subjects suffering from Alzheimer's Disease.

15. A use according to claim 10 wherein the pharmaceutical preparation is for reducing neural damage in subjects who have suffered an ischemic, anoxic or hypoxic insult.

16. A use according to claim 10 wherein the pharmaceutical preparation is for reducing neural damage in subjects who have suffered an ischemic, anoxic or hypoxic insult as a r4esult of a) ischemic stroke, b) hemorrhagic stroke, c) cardiac arrest and resuscitation, d) carbon monoxide poisoning, e) trauma, f) asphyxiation, g) strangulation, h) drowning, i) hemorrhagic shock, j) inhalant substance abuse or huffing, k) brain edema or l) iatrogenic disruption of cerebral circulation during a surgery or other medical procedure.
